(19) United States
(12) Patent Application Publication
(10) Pub. No.: US 2007/0099851 A1
(43) Pub. Date: May 3, 2007

(54) Stable analogues of ribose-1-phosphate and methods for treating diabetes and other metabolic disorders

(76) Inventor: Gregory Saul Linn, New York, NY (US)

Correspondence Address: Gregory Linn, Apt. 35, 414 WEST 121st STREET, New York, NY, 10027, US

(21) Appl. No.: 11/321,570

(22) Filed: Dec. 29, 2005

Related U.S. Application Data
(60) Provisional application No. 60/640,133, filed on Dec. 30, 2004.

(51) Int. Cl. A61K 31/7024; A61K 31/70; C07H 19/04

(52) U.S. Cl. 514/25; 536/26.1

(57) ABSTRACT

Novel D-ribose-1-phosphate analogue compounds of formula I, pharmaceutically acceptable prodrugs and salts thereof, and their use as hypoglycemic agents and anticancer agents and regulators of carbohydrate metabolism are useful for the treatment of diabetes in humans and for the treatment of various metabolic disorders that involve the regulation of cellular metabolism, e.g. cancer

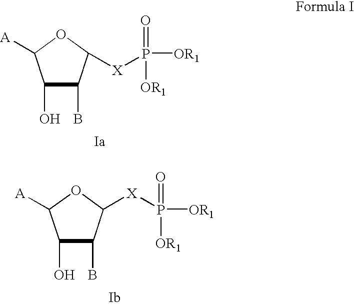

### BACKGROUND OF THE INVENTION

Diabetes mellitis is known to affect approximately 14 million adults in the United States alone. There are two types of this disease: one is Type I or insulin dependent diabetes mellitus (IDDM) and the other is Type II or non-insulin dependent diabetes mellitis (NIDDM). This disease is characterized by hyperglycemia both in the fasted state and post-prandial increase in blood glucose levels. Additionally, diabetes has been associated with many health problems like neuropathy, retinopathy and coronary heart disease. The ability to control blood glucose levels in a diabetic patient would serve to ameliorate the effects of hyperglycemia and benefit the long-term health of these patients.

Currently, the main defects that account for elevated levels of blood glucose are decreased insulin secretion from beta cells of the pancreas, resistance to insulin-mediated uptake of blood glucose by muscle cells and uncontrolled gluconeogenesis in the liver and to a lesser extent in the kidneys caused at least in part by resistance to the uptake of insulin. Anti-diabetic agents that could treat these defects of NIDDM would contribute to the control of diabetes to the benefit of those in need of such intervention.

Glucose utilization, namely the break down of glucose in the process known as glycolysis and the de novo biosynthesis of glucose known as gluconeogenesis are important metabolic pathways and directly affect the diabetic condition in humans. The understanding of their metabolic control has been enhanced by the discovery that some sugar bisphosphate compounds like fructose-2,6-bisphosphate and ribose-1,5-bisphosphate activate glycolysis by stimulating 6-phosphofructokinase and deactivate gluconeogenesis by inhibiting fructose-1,6-bisphosphatase.

5-phosphoribosyl-1-methylenephosphonate, corresponding to compound Ia (X═CH₂, A=CH₂OP(O)(OH)₂, B═OH, R1=H) has been shown to activate 6-phosphofructo-1-kinase with a Ka=6.5 μM and inhibit fructose-1,6-bisphosphatase with a Ki=85 μM. Further it has been shown that such a phosphorylated compound can enter into cultured hepatoma cells (FAO) and inhibit glucose production. Thus it is an object of this invention to provide stable, isosteric compounds related in structure to ribose-1-phosphate, a precursor of ribose-1,5-bisphosphate, which will possess similar biological activity and can be phosphorylated at the C(5) hydroxy in either outside or inside a cell in order to attain a structure like that of ribose-1,5-bisphosphate, which are readily synthesized.

### BRIEF DESCRIPTION OF THE DRAWING

FIG. 1 shows the results of a glucose tolerance test done with compounds 1.1 and 3.1

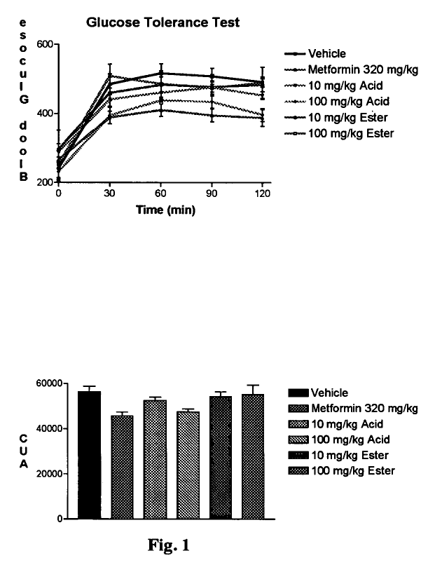

### SUMMARY OF THE INVENTION

Novel isosteric and stable anomers of ribose-1-phosphate of the following formula I 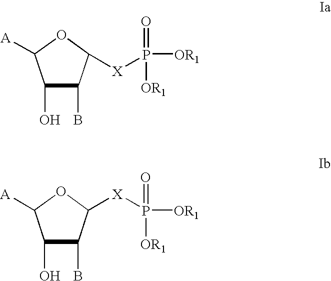
have been shown to inhibit glucose production in cultured cells and in vivo. These compounds have also been shown to inhibit some cancer cell lines. Thus the compounds of this invention are directed toward the use in humans as a method of treating diabetes and cancer. These compounds, as analogues of ribose-1-phosphate, a metabolite occurring in cells, are also directed toward ameliorating the effects of ischemia, and other diseases for which increased levels of ribose-1-phosphate and its subsequent metabolite, ribose-1,5-bisphosphate have been shown to decrease. These compounds are also directed to be treatments for metabolic disorders, which are responsive to lowered blood glucose levels or to the inhibition of gluconeogenesis.

In another aspect these compounds are also useful in inhibiting the growth of cancer cells.

Ribose-1-phosphate (Rib-1-P) and its further metabolite, ribose-1,5-bisphosphate (Rib-P₂) are important regulators of cellular metabolism. Rib-1-P is produced by the phosphorolysis of inosine and related nucleosides. Rib-1-P is subsequently used to help make DNA or is shuttled into the pentose phosphate pathway to provide carbon for glycolytic intermediates. Additionally, Rib-P₂ levels have been shown to greatly increase during ischemia in brain cell and may function to protect cells from ischemic damage. Also, Rib-P₂ has been shown to activate 6-phosphofructokinase and inhibit fructose-1,6-bisphosphatase. Both enzymes are involved in regulating carbohydrate metabolism in liver.

Since ribose-1-phosphate is quite labile in a biological system, being subject to enzyme or general acid/base hydrolysis, a stable version of it would be desirable. Such a compound could be used to provide a ribose-1-phosphate mimic that would function like the naturally occurring compound. Replacing the bridge oxygen of ribose-1-phosphate with CH₂, CHF, CF₂ or some other suitable chemical group would provide for a non-hydrolyzable analogue of ribose-1-phosphate. It is possible that this stable form of ribose-1-phosphate could be phosphorylated in a cell to become a stable analogue of ribose-1,5-bisphosphate. Additionally, it has been shown that 5-iodo-ribose-1-phosphate inhibits human purine nucleoside phosphorylase. Thus compounds of formula I in which the C(5) hydroxy is substituted by halogens or other electron-rich groups increase the efficacy of bonding to an enzyme. Further, substitutions of F at the C(2) carbon for the hydroxy group are known to confer significant changes on the electronic character of a molecule while retaining many of the characteristics of hydroxy group, namely its hydrogen bonding character and its general bond length (1.43 Å C—F vs. 1.35 Å C—O).

### DETAILED DESCRIPTION OF THE INVENTION

### DEFINITIONS

In accordance with the present invention and as used herein, the following terms are defined with the following meanings, unless explicitly stated otherwise.

The term ‘biological system’ includes mammalian or plant cells, and living organisms. Such organisms include but are not limited to humans, animals and bacteria and viruses.

The term “aryl” refers to aromatic groups which have at least one ring having a conjugated pi electron system and includes carbocyclic aryl, heterocyclic aryl and biaryl groups, all of which may be optionally substituted.

Carbocyclic aryl groups are groups wherein the ring atoms on the aromatic ring are carbon atoms. Carbocyclic aryl groups include monocyclic carbocyclic aryl groups and polycyclic or fused compounds such as optionally substituted naphthyl groups.

Heterocyclic aryl groups are groups having from 1 to 4 heteroatoms as ring atoms in the aromatic ring and the remainder of the ring atoms being carbon atoms. Suitable heteroatoms include oxygen, sulfur, and nitrogen. Suitable heteroaryl groups include furanyl, thienyl, pyridyl, pyrrolyl, N-lower alkyl pyrrolyl, pyridyl-N-oxide, pyrimidyl, pyrazinyl, imidazolyl, and the like, all optionally substituted.

The term “biaryl” represents aryl groups containing more than one aromatic ring including both fused ring systems and aryl groups substituted with other aryl groups.

The term “alicyclic” means compounds which combine the properties of aliphatic and cyclic compounds and include but are not limited to aromatic, cycloalkyl and bridged cycloalkyl compounds. The cyclic compound includes heterocycles. Cyclohexenylethyl, cyclohexanylethyl, and norbornyl are suitable alicyclic groups. Such groups may be optionally substituted.

The term “optionally substituted” or “substituted” includes groups substituted by one to four-substituents, independently selected from lower alkyl, lower aryl, lower aralkyl, lower alicyclic, hydroxy, lower alkoxy, lower aryloxy, perhaloalkoxy, aralkoxy, heteroaryl, heteroaryloxy, heteroarylalkyl, heteroaralkoxy, azido, amino, guanidino, halogen, lower alkylthio, oxa, ketone, carboxy esters, carboxyl, carboxamido, nitro, acyloxy, alkylamino, aminoalkyl, alkylaminoaryl, alkylaryl, alkylaminoalkyl, alkoxyaryl, arylamino, aralkylamino, phosphonate, sulfonate, carboxamidoalkylaryl, carboxamidoaryl, hydroxyalkyl, haloalkyl, alkylaminoalkylcarboxy, aminocarboxamidoalkyl, cyano, lower alkoxyalkyl, and lower perhaloalkyl.

The term “aralkyl” refers to an alkyl group substituted with an aryl group. Suitable aralkyl groups include benzyl, picolyl, and the like, and may be optionally substituted.

The term “lower” referred to herein in connection with organic radicals or compounds respectively defines such as with up to and including 10, preferably up to and including 6, and advantageously one to four carbon atoms. Such groups may be straight chain, branched, or cyclic.

The terms “arylamino” (a), and “aralkylamino” (b), respectively, refer to the group —NRR′ wherein respectively, (a) R is aryl and R′ is hydrogen, alkyl, aralkyl or aryl, and (b) R is aralkyl and R′ is hydrogen or aralkyl, aryl, alkyl.

The term “acyl” refers to —C(O)R where R is alkyl and aryl.

The term “carboxy esters” refers to —C(O)OR where R is alkyl, aryl, aralkyl, and alicyclic, all optionally substituted.

The term “oxa” refers to ═O in an alkyl group

The term “alkylamino” refers to —NRR′ where R and R′ are independently selected from hydrogen or alkyl.

The term “carbonylamine” or “carbonylamino” refers to —CONR₂ where each R is independently hydrogen or alkyl

The term “halogen” or “halo” refers to —F, —Cl, —Br and —I.

The term “oxyalkylamino” refers to —O-alk-NR—, where “alk” is an alkylene group and R is H or alkyl.

The term “alkylsulfonate” refers to the group -alk-S(O)2-O— where “alk” is an alkylene group.

The term “alkylaminoalkylcarboxy” refers to the group -alk-NR-alk-C(O)—O— where “alk” is an alkylene group, and R is a H or lower alkyl.

The term “alkylaminocarbonyl” refers to the group -alk-NR-C(O)— where “alk” is an alkylene group, and R is a H or lower alkyl.

The term “oxyalkyl” refers to the group —O-alk- where “alk” is an alkylene group.

The term “alkylcarboxyalkyl” refers to the group -alk-C(O)—O-alkyl where each alk is independently an alkylene group.

The term “alkyl” refers to saturated aliphatic groups including straight-chain, branched chain and cyclic groups. Alkyl groups may be optionally substituted.

The term “bidentate” refers to an alkyl group that is attached by its terminal ends to the same atom to form a cyclic group. For example, propylene imine contains a bidentate propylene group.

The term “cyclic alkyl” refers to alkyl groups that are cyclic.

The term “heterocyclic” and “heterocyclic alkyl” refer to cyclic alkyl groups containing at least one heteroatom. Suitable heteroatoms include oxygen, sulfur, and nitrogen. Heterocyclic groups may be attached through a heteroatom or through a carbon atom in the ring.

The term “alkenyl” refers to unsaturated groups which contain at least one carbon-carbon double bond and includes straight-chain, branched-chain and cyclic groups. Alkene groups may be optionally substituted.

The term “alkynyl” refers to unsaturated groups which contain at least one carbon-carbon triple bond and includes straight-chain, branched-chain and cyclic groups. Alkyne groups may be optionally substituted.

The term “alkylene” refers to a divalent straight chain, branched chain or cyclic saturated aliphatic radical.

The term “acyloxy” refers to the ester group —O—C(O)R, where R is H, alkyl, alkenyl, alkynyl, aryl, aralkyl, or alicyclic.

The term “alkylaryl” refers to the group -alk-aryl- where “alk” is an alkylene group.

“Lower alkylaryl” refers to such groups where alkylene is lower alkyl.

The term “alkylamino” refers to the group -alk-NR— wherein “alk” is an alkylene group.

The term “alkyl(carboxyl)” refers to carboxyl substituted off the alkyl chain. Similarly, “alkyl(hydroxy)”, “alkyl(phosphonate)”, and “alkyl(sulfonate)” refers to substituents off the alkyl chain.

The term “alkylaminoalkyl” refers to the group -alk-NR-alk- wherein each “alk” is an independently selected alkylene, and R is H or lower alkyl. “Lower alkylaminoalkyl” refers to groups where each alkylene group is lower alkyl.

The term “alkylaminoaryl” refers to the group -alk-NR-aryl- wherein “alk” is an alkylene group. In “lower alkylaminoaryl”, the alkylene group is lower alkyl.

The term “alkyloxyaryl” refers to an alkylene group substituted with an aryloxy group. In “lower alkyloxyaryl”, the alkylene group is lower alkyl.

The term “alkylacylamino” refers to the group -alk-N—(COR)— wherein alk is alkylene and R is lower alkyl. In “lower alkylacylamino”, the alkylene group is lower alkyl.

The term “alkoxyalkylaryl” refers to the group -alk-O-alk-aryl- wherein each “alk” is independently an alkylene group. “Lower aloxyalkylaryl” refers to such groups where the alkylene group is lower alkyl.

The term “alkylacylaminoalkyl” refers to the group -alk-N—(COR)-alk- where each alk is an independently selected alkylene group. In “lower alkylacylaminoalkyl” the alkylene groups are lower alkyl.

The term “alkoxy” refers to the group -alk-O— wherein alk is an alkylene group.

The term “alkoxyalkyl” refers to the group -alk-O-alk- wherein each alk is an independently selected alkylene group. In “lower alkoxyalkyl”, each alkylene is lower alkyl.

The term “alkylthio” refers to the group -alk-S— wherein alk is alkylene group.

The term “alkylthioalkyl” refers to the group -alk-S-alk- wherein each alk is an independently selected alkylene group. In “lower alkylthioalkyl” each alkylene is lower alkylene.

The term “aralkylamino” refers to an amine substituted with an aralkyl group.

The term “alkylcarboxamido” refers to the group -alk-C(O)N(R)— wherein alk is an alkylene group and R is H or lower alkyl.

The term “alkylcarboxamidoalkyl” refers to the group -alk-C(O)N(R)-alk- wherein each alk is an independently selected alkylene group and R is lower alkyl. In “lower alkylcarboxamidoalkyl” each alkylene is lower alkyl.

The term “alkylcarboxamidoalkylaryl” refers to the group -alk1-C(O)—NH-alk2Ar— wherein alk1 and alk2 are independently selected alkylene groups and alk2 is substituted with an aryl group, Ar. In “lower alkylcarboxamidoalkylaryl”, each alkylene is lower alkyl.

The term “heteroalicyclic” refers to an alicyclic group having 1 to 4 heteroatoms selected from nitrogen, sulfur, phosphorus and oxygen.

The term “aminocarboxamidoalkyl” refers to the group —NH—C(O)—N(R)—R wherein each R is an independently selected alkyl group. “Lower aminocaboxamidoalkyl” refers to such groups wherein each R is lower alkyl.

The term “heteroarylalkyl” refers to an alkyl group substituted with a heteroaryl group.

The term “perhalo” refers to groups wherein every C—H bond has been replaced with a C-halo bond on an aliphatic or aryl group. Suitable perhaloalkyl groups include —CF₃ and —CFCl₂.

The term “guanidine” refers to both —NR—C(NR)—NR₂ as well as —N═C(NR₂)₂ where each R group is independently selected from the group of —H, alkyl, alkenyl, alkynyl, aryl, and alicyclic, all optionally substituted.

The term “amidine” refers to —C(NR)—NR₂ where each R group is independently selected from the group of —H, alkyl, alkenyl, alkynyl, aryl, and alicyclic, all optionally substituted.

The term “pharmaceutically acceptable salt” includes salts of compounds of formula I and its prodrugs derived from the combination of a compound of this invention and an organic or inorganic acid or base.

The term “prodrug” as used herein refers to any compound that when administered to a biological system generates the “drug” substance either as a result of spontaneous chemical reaction(s) or by enzyme catalyzed or metabolic reaction(s). Reference is made to various prodrugs such as acyl esters, carbonates, and carbamates, included herein. The groups illustrated are exemplary, not exhaustive, and one skilled its the art could prepare other known varieties of prodrugs. Such prodrugs of the compounds of formula I, fall within the scope of the present invention.

The term “prodrug ester” as employed herein includes but is not limited to, the following groups and combinations of these groups.

[1] Acyloxyalkyl esters, which are well described in the literature (Farquhar et al., J. Pharm. Sci. 72,324-325 (1983)) and are represented by formula A

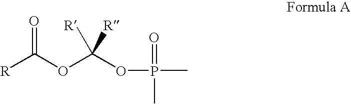

wherein R, R_, and R″ are independently H, alkyl, aryl, alkylaryl, and alicyclic; (see WO 90/08155, WO 90/10636).

Other acyloxyalkyl esters are possible in which an alicyclic ring is formed such as shown in formula B. These esters have been shown to generate phosphorus-containing nucleotides inside cells through a postulated sequence of reactions beginning with deesterification and followed by a series of elimination reactions (e.g. Freed et al., Biochem. Pharm. 38: 3193-3198 (1989)).

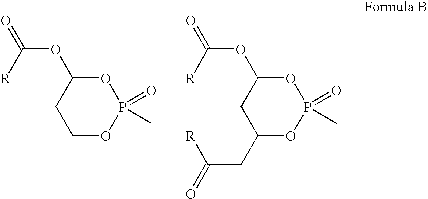

wherein R is —H, alkyl, aryl, alkylaryl, alkoxy, aryloxy, alkylthio, arylthio, alkylamino, arylamino, cycloalkyl, or alicyclic.

[3] Another class of these double esters known as alkyloxycarbonyloxymethyl esters, as shown in formula A, where R is alkoxy, aryloxy, alkylthio, arylthio, alkylamino, and arylamino; R′, and R″ are independently H, alkyl, aryl, alkylaryl, and alicyclic, have been studied in the area of β-lactam antibiotics (Tatsuo Nishimura et al. J. Antibiotics, 1987, 40(1), 81-90; for a review see Ferres, H., Drugs of Today, 1983,19, 499.). More recently Cathy, M. S., et al. (Abstract from AAPS Western Regional Meeting, April, 1997) showed that these alkyloxycarbonyloxymethyl ester prodrugs on (9-[(R)-2-phosphonomethoxy)propyl]adenine (PMPA) are bioavailable up to 30% in dogs.

[4] Aryl esters have also been used as phosphonate prodrugs (e.g. Erion, DeLambert et al., J. Med. Chem. 37. 498, 1994; Serafinowska et al., J. Med. Chem. 38: 1372, 1995).

Phenyl as well as mono and poly-substituted phenyl phosphonate ester prodrugs have generated the parent phosphonic acid in studies conducted in animals and in man (Formula C). Another approach has been described where Y is a carboxylic ester ortho to the phosphate. Khamnei and Torrence, J. Med. Chem.; 39:4109-4115 (1996).

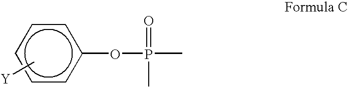

wherein Y is H, alkyl, aryl, alkylaryl, alkoxy, acetoxy, halogen, amino, alkoxycarbonyl, hydroxy, cyano, alkylamino, and alicyclic.

[5] Benzyl esters have also been reported to generate the parent phosphonic acid. In some cases, using substituents at the para-position can accelerate the hydrolysis. Benzyl analogs with 4-acyloxy or 4-alkyloxy group [Formula D, X═H, OR or O(CO)R or O(CO)OR] can generate the 4-hydroxy compound more readly through the action of enzymes, e.g,. oxidases, esterases, etc. Examples of this class of prodrugs are described by Mitchell et al., J. Chem. Soc. Perkin Trans. [2345 (1992); Brook, et al. WO 91/19721.

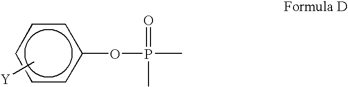

wherein X and Y are independently H, alkyl, aryl, alkylaryl, alkoxy, acetoxy, hydroxy, cyano, nitro, perhaloalkyl, halo, or alkyloxycarbonyl; and
R′ [symbol] and R″ are independently H, alkyl, aryl, alkylaryl, halogen, and alicyclic.

[6] Thio-containing phosphonate phosphonate ester prodrugs have been described that are useful in the delivery of prodrugs to hepatocytes. These phosphonate ester prodrugs contain a protected thioethyl moiety as shown in formula E. One or more of the oxygens of the phosphonate can be esterified. Since the mechanism that results in de-esterification requires the generation of a free thiolate, a variety of thiol protecting groups are possible. For example, the disulfide is reduced by a reductase-mediated process (Puech et al., Antiviral Res., 22: 155-174 (1993)). Thioesters will also generate free thiolates after esterase-mediated hydrolysis. Benzaria, et al., J. Med. Chem., 39:4958 (1996). Cyclic analogs are also possible and were shown to liberate phosphonate in isolated rat hepatocytcs. The cyclic disulfide shown below has not been previously described and is novel.

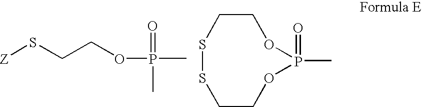

wherein Z is alkylcarbonyl, alkoxycarbonyl, arylcarbonyl, aryloxycarbonyl, or alkylthio.

Other examples of suitable prodrugs include proester classes exemplified by Biller and Magnin (U.S. Pat. No. 5,157,027); Serafinowska et al. (J. Med. Chem. 38, 1372 (1995)); Starrett et al. (J. Med. Chem. 37, 1857 (1994)); Martin et al. J. Pharm. Sci. 76, 180 (1987); Alexander et al., Collect. Czech. Chem. Commun, 59, 1853 (1994)); and EPO patent application 0 632 048 A1. Some of the structural classes described are optionally substituted, including fused lactones attached at the omega position and optionally substituted 2-oxo-1,3-dioxolenes attached through a methylene to the phosphorus oxygen such as:

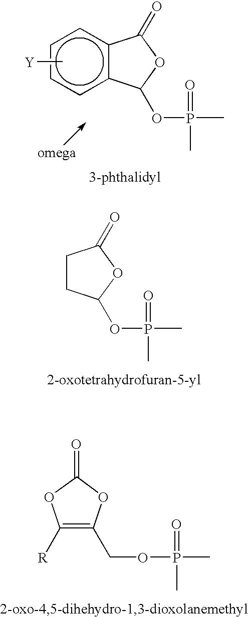

2-oxo-4,5-didehydro-1,3-3-phthalidyl 2-oxotetrahydrofuran-5-yl dioxolanemethyl

wherein R is —H, alkyl; cycloalkyl, or alicyclic; and

wherein Y is —H, alkyl, aryl, alkylaryl, cyano, alkoxy, acetoxy, halogen, amino, alkylamino, alicyclic, and alkoxycarbonyl.

[7] Propyl phosphonate ester prodrugs can also be used to deliver prodrugs into hepatocytes. These phosphonate ester prodrugs may contain a hydroxyl and hydroxyl group derivatives at the 3-position of the propyl group as shown in formula F. The R and X groups can form a cyclic ring system as shown in formula F. One or more of the oxygens of the phosphonate can be esterified.

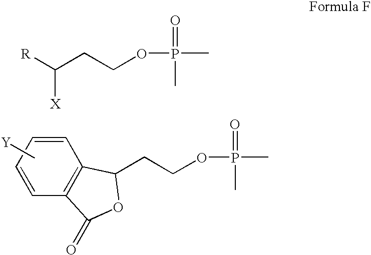

wherein R is alkyl, aryl, heteroaryl;
X is hydrogen, alkylcarbonyloxy, alkyloxycarbonyloxy; and
Y is alkyl, aryl, heteroaryl, alkoxy, alkylamino, alkylthio, halogen, hydrogen, hydroxy, acetoxy, amino.

[8] The cyclic propyl phosphonate esters as in Formula G are shown to activate to phosphonic acids. The activation of prodrug can be mechanistically explained by in vivo oxidation and elimination steps. These prodrugs inhibit glucose production in isolated rat hepatocytes and are also shown to deliver prodrugs to the liver following oral administration.

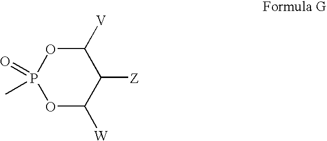

wherein

V and W are independently selected from the group consisting of hydrogen, aryl, substituted aryl, heteroaryl, substituted heteroaryl, 1-alkenyl, 1-alkynyl, and —R⁹; or

together V and Z are connected to form a cyclic group containing 3-5 atoms, optionally 1 heteroatom, substituted with hydroxy, acyloxy, alkoxycarboxy, or aryloxycarboxy attached to a carbon atom that is three atoms from an oxygen attached to the phosphorus; or

together V and W are connected to form a cyclic group containing 3 carbon atoms substituted with hydroxy, acyloxy, alkoxycarboxy, alkylthiocarboxy, hydroxymethyl, and aryloxycarboxy attached to a carbon atom that is three atoms from an oxygen attached to the phosphorus;

Z is selected from the group consisting of —CH₂OH, —CH₂OCOR³, —CH₂OC(O)R³, —CH₂OC(O)SR³, —CH₂OCO₂R³, —SR³, —S(O)R³, —CH₂N₃, —CH₂NR²₂, —CH₂Ar, —CH(Ar)OH, —CH(CH═CR²R²)OH, —CH(C═CR²)OH, and —R²; with the provisos that:

a) V, Z, W are not all —H; and

b) when Z is —R², then at least one of V and W is not —H or —R⁹;

R² is selected from the group consisting of R³ and —H;

R³ is selected from the group consisting of alkyl, aryl, alicyclic, and aralkyl; and

R⁹ is selected from the group consisting of alkyl, aralkyl, and alicyclic.

[9] Phosphoramidate derivatives have been explored as potential phosphonate prodrugs (e.g. McGuigan et al., Antiviral Res. 1990, 14: 345; 1991, 15: 255. Serafinowska et al., J. Med. Chem., 1995, 38, 1372). Most phosphoramidates are unstable under aqueous acidic conditions and are hydrolyzed to the corresponding phosphonic acids. Cyclic phosphoramidates have also been studied as phosphonate prodrugs because of their potential for greater stability compared to non cyclic phosphoramidates (e.g. Starrett et al., J. Med. Chem., 1994, 37: 1857).

Other prodrugs are possible based on literature reports such as substituted ethyls for example, bis(trichloroethyl)esters as disclosed by McGuigan, et al. Bioorg Med. Chem. Let., 3:1207-1210 (1993), and the phenyl and benzyl combined nucleotide esters reported by Meier, C. et al. Bioorg. Med. Chem. Lett., 7:99-104 (1997).

X group nomenclature as used herein in formula I describes this group attached to the phosphonate and ends with this group attached to the C(1) position of the ribose ring.

### DETAILED DESCRIPTION OF THE INVENTION

Novel Analogues of Ribose-1-Phosphate

Preferred compounds of the present invention are inhibitors of gluconeogenesis, stimulators of insulin secretion and can relieve insulin resistance in cells. These compounds are also inhibitors of cancer cell growth and are of formula I:

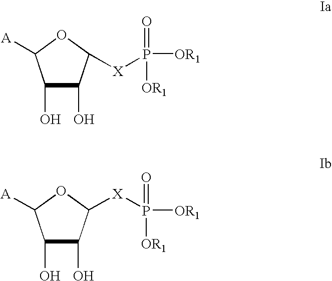

wherein X is selected from the group consisting of CH₂, CHF, CF₂, S NH, alkyl, alkenyl, alkynyl, alkyl(carboxyl), alkyl(hydroxy), alkyl(phosphonate), alkyl(sulfonate, aryl, alkylaminoalkyl, alkoxyalkyl, alkylthioalkyl, alkylthio, alicyclic, 1-monohaloalkyl, 1,1 dihaloalkyl, carbonylalkyl, aminocarbonylamino, alkylaminocarbonyl, alkylcarbonylamino, aralkyl and alkylaryl, all optionally substituted; A is (CH₂)ₙ-A′ where n is from 1-4 and A′ is hydroxy, halogen, phosphate, alkyl, alkoxy, amino, azido or alkenyl; B is OH or F. R1 is independently selected from the group consisting of alkyl, aryl, heteroalicyclic where the cyclic moiety contains a carbonate or thiocarbonate, —C(R²)₂-aryl, -alk-aryl, —C(R²)₂ OC(O)NR²₂, —NR²—C(O)—R³, —C(R²)₂—OC(O)R³, —C(R²)₂—O—C(O)OR³, —C(R²)₂ OC(O)SR³, -alk-S—C(O)R³, -alk-S—S-alkylhydroxy, and -alk-S—S—S-alkylhydroxy, or together R1 and R1 are -alk-S—S-alk- to form a cyclic group, wherein each “alk” is an independently selected alkylene, or together R1 and R1 are

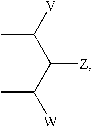

wherein

V and W are independently selected from the group consisting of hydrogen, aryl, substituted aryl, heteroaryl, substituted heteroaryl, 1-alkenyl, 1-alkynyl, and —R⁹; or

together V and Z are connected via a chain of 3-5 atoms, only one of which can be a heteroatom, to form part of a cyclic group substituted with hydroxy, acyloxy, alkoxycarboxy, or aryloxycarboxy attached to a carbon atom that is three atoms from an oxygen attached to the phosphorus; or

together V and W are connected via a chain of 3 carbon atoms to form part of a cyclic group substituted with hydroxy, acyloxy, alkoxycarboxy, alkylthiocarboxy, hydroxymethyl, or aryloxycarboxy attached to a carbon atom that is three atoms from an oxygen attached to the phosphorus;

Z is selected from the group consisting of —CH₂OH, —CH₂OCOR³, —CH₂OC(O)R³, —CH₂OC(O)SR³, —CH₂OCO₂R³, —SR³, —S(O)R³, —CH₂ N₃, —CH₂ NR²₂, —CH₂Ar, —CH(Ar)OH, —CH(CH═CR²R²)OH, —CH(C═CR²)OH, and —R²;

with the provisos that:

a) V, Z, W are not all —H; and

b) when Z is —R², then at least one of V and W is not —H or —R⁹;

R² is selected from the group consisting of R³ and —H;

R³ is selected from the group consisting of alkyl aryl, alicyclic, heteroalicyclic, and aralkyl;

R⁴ is independently selected from the group consisting of —H, lower alkyl, lower alicyclic, lower heteroalicyclic, lower aralkyl, and lower aryl;

R⁵ is selected from the group consisting of lower alkyl, lower aryl, lower aralkyl, lower alicyclic, and lower heteroalicyclic;

R⁶ is independently selected from the group consisting of —H, and lower alkyl;

R⁷ is independently selected from the group consisting of —H, lower alkyl, lower alicyclic, lower heteroalicyclic, lower aralkyl, lower aryl, and —C(O)R¹⁰;

R⁸ is independently selected from the group consisting of —H, lower alkyl, lower aralkyl, lower aryl, lower alicyclic, —(O)R¹⁰, or together said R⁸ groups form a bidendate alkylene;

R⁹ is selected from the group consisting of alkyl, aralkyl, alicyclic, and heteroalicyclic;

R¹⁰ is selected from the group consisting of —H, lower alkyl, —NH₂, lower aryl, and lower perhaloalkyl;

R¹¹ is selected from the group consisting of alkyl, aryl, —OH, —NH₂ and —OR³; and pharmaceutically acceptable prodrugs and salts thereof.

Preferred Compounds of Formula 1

Suitable alkyl groups include groups having from 1 to about 20 carbon atoms. Suitable aryl groups include groups having from 1 to about 20 carbon atoms. Suitable aralkyl groups include groups having from 2 to about 21 carbon atoms. Suitable acyloxy groups include groups having from 1 to about 20 carbon atoms. Suitable alkylene groups include groups having from 1 to about 20 carbon atoms. Suitable alicyclic groups include groups having 3 to about 20 carbon atoms. Suitable heteroaryl groups include groups having from 1 to about 20 carbon atoms and from 1 to 5 heteroatoms, preferably independently selected from nitrogen, oxygen, phosphorous, and sulfur. Suitable heteroalicyclic groups include groups having from 2 to about twenty carbon atoms and from 1 to 5 heteroatoms, preferably independently selected from nitrogen, oxygen, phosphorous, and sulfur.

Preferred X groups include 1-haloalkyl, 1,1 dihaloalkyl, alkyl, amino, alkylamino, thio, alkylthio. Particularly preferred is alkyl substituted with 1 to 3 substituents selected from halogen, phosphonate, —CO₂H, —SO₃H, and —OH. Particularly preferred 1-haloalkyl groups is fluoromethyl. Particularly preferred 1,1-dihaloalkyl groups is difluoromethyl. Particularly preferred alkyl is methyl.

Preferred A groups include A=CH₂-A′ and A′ is hydroxy, halogen, phosphate, alkyl, alkenyl.

Preferred B groups include B is OH or F.

Preferred R1 is independently selected from the group consisting of alkyl, aryl, heteroalicyclic where the cyclic moiety contains a carbonate or thiocarbonate, —C(R²)₂-aryl, -alk-aryl, —C(R²)₂OC(O)NR²₂, —NR²—C(O)—R³, —C(R²)₂—OC(O)R³, —C(R²)₂—O—C(O)OR³, —C(R²)₂OC(O)SR³, -alk-S—C(O)R³, -alk-S—S-alkylhydroxy, and -alk-S—S—S-alkylhydroxy, or together R1 and R1 are -alk-S—S-alk- to form a cyclic group, wherein each “alk” is an independently selected alkylene, or together R1 and R1 are

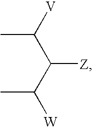

wherein

V and W are independently selected from the group consisting of hydrogen, aryl, substituted aryl, heteroaryl, substituted heteroaryl, 1-alkenyl, 1-alkynyl, and —R⁹; or

together V and Z are connected via a chain of 3-5 atoms, only one of which can be a heteroatom, to form part of a cyclic group substituted with hydroxy, acyloxy, alkoxycarboxy, or aryloxycarboxy attached to a carbon atom that is three atoms from an oxygen attached to the phosphorus; or

together V and W are connected via a chain of 3 carbon atoms to form part of a cyclic group substituted with hydroxy, acyloxy, alkoxycarboxy, alkylthiocarboxy, hydroxymethyl, or aryloxycarboxy attached to a carbon atom that is three atoms from an oxygen attached to the phosphorus;

Z is selected from the group consisting of —CH₂ OH, —CH₂OCOR³, —CH₂OC(O)R³, —CH₂OC(O)SR³, —CH₂OCO₂R³, —SR³, —S(O)R³, —CH₂ N₃, —CH₂ NR²₂, —CH₂Ar, —CH(Ar)OH, —CH(CH═CR²R²)OH, —CH(C═CR²)OH, and —R²;

with the provisos that:

a) V, Z, W are not all —H; and

b) when Z is —R², then at least one of V and W is not —H or —R⁹;

R² is selected from the group consisting of R³ and —H;

R³ is selected from the group consisting of alkyl aryl, alicyclic, heteroalicyclic, and aralkyl;

R⁴ is independently selected from the group consisting of —H, lower alkyl, lower alicyclic, lower heteroalicyclic, lower aralkyl, and lower aryl;

R⁵ is selected from the group consisting of lower alkyl, lower aryl, lower aralkyl, lower alicyclic, and lower heteroalicyclic;

R⁶ is independently selected from the group consisting of —H, and lower alkyl;

R⁷ is independently selected from the group consisting of —H, lower alkyl, lower alicyclic, lower heteroalicyclic, lower aralkyl, lower aryl, and —C(O)R¹⁰;

R⁸ is independently selected from the group consisting of —H, lower alkyl, lower aralkyl, lower aryl, lower alicyclic, —(O)R¹⁰, or together said R⁸ groups form a bidendate alkylene;

R⁹ is selected from the group consisting of alkyl, aralkyl, alicyclic, and heteroalicyclic;

R¹⁰ is selected from the group consisting of —H, lower alkyl, —NH₂, lower aryl, and lower perhaloalkyl;

R¹¹ is selected from the group consisting of alkyl, aryl, —OH, —NH₂ and —OR³; and pharmaceutically acceptable prodrugs and salts thereof.

Preferred R1 groups include —H, alkylaryl, aryl, —C(R²)₂-aryl, and —C(R²)₂ —OC(O)R³. Preferred such R1 groups include optionally substituted phenyl, optionally substituted benzyl, —H, —C(R²)₂OC(O)OR³, and —C(R2)2OC(O)R3. Preferably, said alkyl groups are greater than 4 carbon atoms. Another preferred aspect is where at least one R1 is aryl or —C(R²)₂-aryl. Also particularly preferred are compounds where R1 is alicyclic where the cyclic moiety contains carbonate or thiocarbonate. Another preferred aspect is when at least one R1 is —C(R²)₂—OC(O)R³, —C(R²)₂—OC(O)OR³ or —C(R²)₂—OC(O)SR³. Also particularly preferred is when R1 and R1 together are optionally substituted, including fused, lactone attached at the omega position or are optionally substituted 2-oxo-1,3-dioxolenes attached through a methylene to the phosphorus oxygen. Also preferred is when at least one R is -alkyl-S—S-alkylhydroxyl, -alkyl-S—C(O)R³, and -alkyl-S—S—S-alkylhydroxy, or together R1 and R1 are -alkyl-S—S-alkyl- to form a cyclic group. Also preferred is where R1 and R1 together are

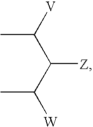

to form a cyclic group, wherein

V and W are independently selected from the group consisting of hydrogen, aryl, substituted aryl, heteroaryl, substituted heteroaryl, 1-alkenyl, 1-alkynyl, and —R⁹; or

together V and Z are connected to form a cyclic group containing 3-5 atoms, optionally 1 heteroatom, substituted with hydroxy, acyloxy, alkoxycarboxy, or aryloxycarboxy attached to a carbon atom that is three atoms from an oxygen attached to the phosphorus; or

together V and W are connected to form a cyclic group containing 3 carbon atoms substituted with hydroxy, acyloxy, alkoxycarboxy, alkylthiocarboxy, hydroxymethyl, and aryloxycarboxy attached to a carbon atom that is three atoms from an oxygen attached to the phosphorus;

Z is selected from the group consisting of —CH₂ OH, —CH₂ OCOR³, —CH₂OC(O)R³, —CH₂OC(O)SR³, —CH₂OCO₂R³, —SR³, —S(O)R³, —CH₂ N₃, —CH₂ NR²₂, —CH₂Ar, —CH(Ar)OH, —CH(CH═CR²R²)OH, —CH(C═CR²)OH, and —R²;

with the provisos that:

a) V, Z, W are not all —H; and

b) when Z is —R², then at least one of V and W is not —H or —R⁹;

R² is selected from the group consisting of R³ and —H;

R³ is selected from the group consisting of alkyl, aryl, alicyclic, and aralkyl; and

R⁹ is selected from the group consisting of alkyl, aralkyl, and alicyclic.

Particularly preferred are such groups wherein V and W both form a 6-membered carbocyclic ring substituted with 0-4 groups, selected from the group consisting of hydroxy, acyloxy alkoxycarbonyl, and alkoxy; and Z is R2. Also particularly preferred are such groups wherein V and W are hydrogen; and Z is selected from the group consisting of hydroxyalkyl, acyloxyalkyl, alkyloxyalkyl, and alkoxycarboxyalkyl. Also particularly preferred are such groups wherein V and W are independently selected from the group consisting of hydrogen, optionally substituted aryl, and optionally substituted heteroaryl, with the proviso that at least one of V and W is optionally substituted aryl or optionally substituted heteroaryl.

In one preferred aspect, R1 is not lower alkyl of 1-4 carbon atoms.

Esters:

In the following examples of preferred compounds, the following prodrugs are preferred:

Acyloxyalkyl esters;

Alkoxycarbonyloxyalkyl esters;

Aryl esters,

Benzyl and substituted benzyl esters;

Disulfide containing esters;

Substituted (1,3-dioxolen-2-one)methyl esters;

Substituted 3-phthalidyl esters;

Cyclic-[2_-hydroxymethyl]-1,3-propanyl diesters and hydroxy protected forms;

Lactone type esters; and all mixed esters resulted from possible combinations of above esters.

Bis-pivaloyloxymethyl esters;

Bis-isobutyryloxymethyl esters;

Cyclic-[2_-hydroxymethyl]-1,3-propanyl diester;

Cyclic-[2_-acetoxymethyl]-1,3-propanyl diester;

Cyclic-[2_-methyloxycarbonyloxymethyl]-1,3-propanyl diester;

Bis-benzoylthiomethyl esters;

Bis-benzoylthioethyl esters;

Bis-benzoyloxymethyl esters;

Bis-p-fluorobenzoyloxymethyl esters;.

Bis-6-chloronicotinoyloxymethyl esters;

Bis-5-bromonicotinoyloxymethyl esters;

Diethyl esters

Bis-thiophenecarbonyloxymethyl esters;

Bis-2-furoyloxymethyl esters;

Bis-3-furoyloxymethyl esters;

Diphenyl esters;

Bis-(4-methoxyphenyl)esters;

Bis-(2-methoxyphenyl)esters;

Bis-(2-ethoxyphenyl)esters;

Mono-(2-ethoxyphenyl)esters;

Bis-(4-acetamidophenyl)esters;

Bis-(4-aceyloxyphenyl)esters;

Bis-(4-hydroxyphenyl)esters;

Bis-(2-acetoxyphenyl)esters;

Bis-(3-acetoxyphenyl)esters;

Bis-(4-morpholinophenyl)esters;

Bis-[4-(1-triazolophenyl)esters;

B is-(3-N,N-dimethylaminophenyl)esters;

Bis-(2-tetrahydronapthyl)esters;

Bis-(3-chloro-4-methoxy)benzyl esters;

Bis-(3-bromo-4-methoxy)benzyl esters;

Bis-(3-cyano-4-methoxy)benzyl esters;

Bis-(3-chloro-4-acetoxy)benzyl esters;

Bis-(3-bromo-4-acetoxy)benzyl esters;

Bis-(3-cyano-4acetoxy)benzyl esters;

Bis-(4-chloro)benzyl esters;

Bis-(4-acetoxy)benzyl esters;

Bis-(3,5-dimethoxy-4-acetoxy)benzyl esters;

Bis-(3-methyl-4-acetoxy)benzyl esters;

Bis-(benzyl)esters;

Dimethyl esters

Bis-(3-methoxy-4-acetoxy)benzyl esters;

Bis-(3-chloro-4-acetoxy)benzyl esters;

cyclic-(2,2-dimethylpropyl)phosphonoamidate;

cyclic-(2-hydroxymethylpropyl)ester;

Bis-(6_-hydroxy-3_{—},4_-disulfide)hexyl esters;

Bis-(6_-acetoxy-3_{—},4_-disulfide)hexyl esters;

(3_{—},4_-Dithia)cyclononane esters;

Bis-(5-methyl-1,3-dioxolen-2-one-4-yl)methyl esters;

Bis-(5-ethyl-1,3-dioxolen-2-one-4-yl)methyl esters;

Bis-(5-tert-butyl-1,3-dioxolen-2-one-4-yl)methyl esters;

Bis-3-(5,6,7-trimethoxy)phthalidyl esters;

Bis-(cyclohexyloxycarbonyloxymethyl)esters;

Bis-(isopropyloxycarbonyloxymethyl)esters;

Bis-(ethyloxycarbonyloxymethyl)esters;

Bis-(methyloxycarbonyloxymethyl)esters;

Bis-(isopropylthiocarbonytoxymethyl)esters;

Bis-(phenyloxycarbonyloxymethyl)esters;

Bis-(benzyloxycarbonyloxymethyl)esters;

Bis-(phenylthiocarbonyloxymethyl)esters;

Bis-(p-methoxyphenyloxycarbonyloxymethyl)esters;

Bis-(m-methoxyphenyloxycarbonyloxymethyl)esters;

Bis-(o-methoxyphenyloxycarbonyloxymethyl)esters;

Bis-(o-methylphenyloxycarbonyloxymethyl)esters;

Bis-(p-chlorophenyloxycarbonyloxymethyl)esters;

Bis-(1,4-biphenyloxycarbonyloxymethyl)esters;

Bis-[(2-phthalimidoethyl)oxycarbonyloxymethyl]esters;

Bis-(N-Phenyl,N-methylcarbamoyloxymethyl)esters;

Bis-(2-trichloroethyl)esters;

Bis-(2-bromoethyl)esters;

Bis-(2-iodoethyl)esters;

Bis-(2-azidoethyl)esters;

Bis-(2-acetoxyethyl)esters;

Bis-(2-aminoethyl)esters;

Bis-(2-N,N-diaminoethyl)esters;

Bis-(2-aminoethyl)esters;

Bis-(methoxycarbonylmethyl)esters,

Bis-(2-aminoethyl)esters;

Bis-[N,N-di(2-hydroxyethyl)]amidomethylesters;

Bis-(2-aminoethyl)esters;

Bis-(2-methyl-5-thiozolomethyl)esters;

Bis-(bis-2-hydroxyethylamidomthyl)esters;

Most preferred are the following:

Bis-pivaloyloxymethyl esters;

Bis-isobutyryloxymethyl esters;

cyclic-(2-hydroxymethylpropyl)ester;

cyclic-(2-acetoxymethylpropyl)ester;

cyclic-(2-methyloxycarbonyloxymethylpropyl)ester;

cyclic-(2-cyclohexylcarbonyloxymethylpropyl)ester;

cyclic-(2-aminomethylpropyl)ester;

cyclic-(2-azidomethylpropyl)ester;

Bis-benzoylthiomethyl esters;

Bis-benzoylthioethylesters;

Bis-benzoyloxymethyl esters;

Bis-p-fluorobenzoyloxymethyl esters;

Bis-6-chloronicotinoyloxymethyl esters;

Bis-5-bromonicotinoyloxymethyl esters;

Bis-thiophenecarbonyloxymethyl esters;

Bis-2-furoyloxymethyl esters;

Bis-3-furoyloxymethyl esters;

Diphenyl esters;

Bis-(2-methyl)phenyl esters;

Bis-(2-methoxy)phenyl esters;

Bis-(2-ethoxy)phenyl esters;

Bis-(4methoxy)phenyl esters;

Bis-(3-bromo-4-methoxy)benzyl esters;

Bis-(4-acetoxy)benzyl esters;

Bis-(3,5-dimethoxy-4-acetoxy)benzyl esters;

Bis-(3-methyl-4-acetoxy)benzyl esters;

Bis-(3-methoxy-4-acetoxy)benzyl esters;

Bis-(3-chloro-4-acetoxy)benzyl esters;

Bis-(cyclohexyloxycarbonyloxymethyl)esters;

Bis-(isopropyloxycarbonyloxymethyl)esters;

Bis-(ethyloxycarbonyloxymethyl)esters;

Bis-(methyloxycarbonyloxymethyl)esters;

Bis-(isopropylthiocarbonyloxymethyl)esters;

Bis-(phenyloxycarbonyloxymethyl)esters;

Bis-(benzyloxycarbonyloxymethyl)esters;

Bis-(phenylthiocarbonyloxymethyl)esters;

Bis-(p-methoxyphenyloxycarbonyloxymethyl)esters;

Bis-(m-methoxyphenyloxycarbonyloxymethyl)esters;

Bis-(o-methoxyphenyloxycarbonyloxymethyl)esters;

Bis-(o-methylphenyloxycarbonyloxymethyl)esters;

Bis-(p-chlorophenyloxycarbonyloxymethyl)esters;

Bis-(1,4-biphenyloxycarbonyloxymethyl)esters;

Bis-[(2-phthalimidoethyl)oxycarbonyloxymethyl]esters;

Bis-(6_-hydroxy-3_{—},4_-disulfide)hexyl esters; and

(3_{—},4_-Disulfide)cyclononane esters.

Bis-(2-bromoethyl)esters;

Bis-(2-aminoethyl)esters;

Bis-(2-N,N-diaminoethyl)esters;

Examples of preferred compounds include but are not limited to those described in Table 1 including salts and prodrugs thereof:

TABLE 1

| | X | A | B |
|---|---|---|---|
| 1. | CH₂ | CH₂OH or CH₂NH₂ or CH₂F or CH₂Br or CH₂I or CH₂OPO(OH)₂ | OH or F |
| 2. | CHF | CH₂OH or CH₂NH₂ or CH₂F or CH₂Br or CH₂I or CH₂OPO(OH)₂ | OH or F |
| 3. | CHCl | CH₂OH or CH₂NH₂ or CH₂F or CH₂Br or CH₂I or CH₂OPO(OH)₂ | OH or F |
| 4. | CHBr | CH₂OH or CH₂NH₂ or CH₂F or CH₂Br or CH₂I or CH₂OPO(OH)₂ | OH or F |
| 5. | CHI | CH₂OH or CH₂NH₂ or CH₂F or CH₂Br or CH₂I or CH₂OPO(OH)₂ | OH or F |
| 6. | CF₂ | CH₂OH or CH₂NH₂ or CH₂F or CH₂Br or CH₂I or CH₂OPO(OH)₂ | OH or F |
| 7. | CCl₂ | CH₂OH or CH₂NH₂ or CH₂F or CH₂Br or CH₂I or CH₂OPO(OH)₂ | OH or F |
| 8. | CBr₂ | CH₂OH or CH₂NH₂ or CH₂F or CH₂Br or CH₂I or CH₂OPO(OH)₂ | OH or F |
| 9. | CI₂ | CH₂OH or CH₂NH₂ or CH₂F or CH₂Br or CH₂I or CH₂OPO(OH)₂ | OH or F |
| 10. | CHNH₂ | CH₂OH or CH₂NH₂ or CH₂F or CH₂Br or CH₂I or CH₂OPO(OH)₂ | OH or F |
| 11. | S | CH₂OH or CH₂NH₂ or CH₂F or CH₂Br or CH₂I or CH2OPO(OH)₂ | OH or F |
| 12. | NH | CH₂OH or CH₂NH₂ or CH₂F or CH₂Br or CH₂I or CH2OPO(OH)₂ | OH or F |
| 13. | CH₂CH₂ | CH₂OH or CH₂NH₂ or CH₂F or CH₂Br or CH₂I or CH2OPO(OH)₂ | OH or F |
| 14. | CHFCHF | CH₂OH or CH₂NH₂ or CH₂F or CH₂Br or CH₂I or CH2OPO(OH)₂ | OH or F |
| 15. | CF₂CF₂ | CH₂OH or CH₂NH₂ or CH₂F or CH₂Br or CH₂I or CH₂OPO(OH)₂ | OH or F |
| 16. | CHBrCHBr | CH₂OH or CH₂NH₂ or CH₂F or CH₂Br or CH₂I or CH₂OPO(OH)₂ | OH or F |
| 17. | CBr₂CBr₂ | CH₂OH or CH₂NH₂ or CH₂F or CH₂Br or CH₂I or CH₂OPO(OH)₂ | OH or F |
| 18. | CHICHI | CH₂OH or CH₂NH₂ or CH₂F or CH₂Br or CH₂I or CH₂OPO(OH)₂ | OH or F |
| 19. | CI₂CI₂ | CH₂OH or CH₂NH₂ or CH₂F or CH₂Br or CH₂I or CH₂OPO(OH)₂ | OH or F |
| 20. | CH₂CH(OH) | CH₂OH or CH₂NH₂ or CH₂F or CH₂Br or CH₂I or CH₂OPO(OH)₂ | OH or F |
| 21. | CH₂CH(CH₃) | CH₂OH or CH₂NH₂ or CH₂F or CH₂Br or CH₂I or CH₂OPO(OH)₂ | OH or F |
| 22. | ethyldialkyl | CH₂OH or CH₂NH₂ or CH₂F or CH₂Br or CH₂I or CH₂OPO(OH)₂ | OH or F |
| 23. | CHS-CH₂ | CH₂OH or CH₂NH₂ or CH₂F or CH₂Br or CH₂I or CH₂OPO(OH)₂ | OH or F |
| 24. | CHS-CHS | CH₂OH or CH₂NH₂ or CH₂F or CH₂Br or CH₂I or CH₂OPO(OH)₂ | OH or F |
| 25. | CH₂—S—CH₂ | CH₂OH or CH₂NH₂ or CH₂F or CH₂Br or CH₂I or CH₂OPO(OH)₂ | OH or F |
| 26. | CHNH₂CH₂ | CH₂OH or CH₂NH₂ or CH₂F or CH₂Br or CH₂I or CH₂OPO(OH)₂ | OH or F |
| 27. | CHOCH₃ | CH₂OH or CH₂NH₂ or CH₂F or CH₂Br or CH₂I or CH₂OPO(OH)₂ | OH or F |
| 28. | CH(OCH₃)CH₂ | CH₂OH or CH₂NH₂ or CH₂F or CH₂Br or CH₂I or CH₂OPO(OH)₂ | OH or F |
| 29. | CH(OCH₂CH₃)CH₂ | CH₂OH or CH₂NH₂ or CH₂F or CH₂Br or CH₂I or CH₂OPO(OH)₂ | OH or F |
| 30. | n-proplyhydroxy | CH₂OH or CH₂NH₂ or CH₂F or CH₂Br or CH₂I or CH₂OPO(OH)₂ | OH or F |
| 31. | n-propylhalo | CH₂OH or CH₂NH₂ or CH₂F or CH₂Br or CH₂I or CH₂OPO(OH)₂ | OH or F |
| 32. | n-propyldihalo | CH₂OH or CH₂NH₂ or CH₂F or CH₂Br or CH₂I or CH₂OPO(OH)₂ | OH or F |
| 33. | n-propyltrihalo | CH₂OH or CH₂NH₂ or CH₂F or CH₂Br or CH₂I or CH₂OPO(OH)₂ | OH or F |
| 34. | n-propylthio | CH₂OH or CH₂NH₂ or CH₂F or CH₂Br or CH₂I or CH₂OPO(OH)₂ | OH or F |

In a less preferred form of Formula I

A for the above listed can also be CH₂OCH₃ or CH₂OCH₂CH₃ or CH₂CH═CH₂.

The most preferred forms of Formula I are where A is CH₂OH or CH₂OPO(OH)₂; B is OH; X is CHF or CF₂ and R1 is H or an ester selected from the esters lists.

Alternatively, the most preferred forms of Formula I are where A is CH₂OH; B is OH; X is CH₂ and R1 is an ester selected from the esters lists.

Preparation of Phosphonate Prodrugs:

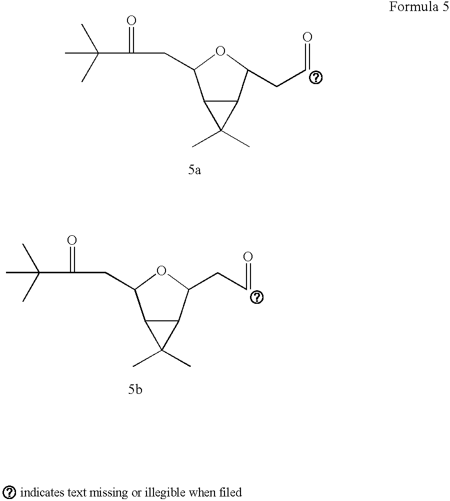

Prodrug esters can be introduced at different stages of the synthesis. Because of their lability, prodrugs are often prepared from compounds of formula 5 where R1 is H. Advantageously, these prodrug esters can be introduced at an early stage, provided that it can withstand the reaction conditions of the subsequent steps.

Compounds of formula I where R1 is H, can be alkylated with electrophiles (such as alkyl halides, alkyl sulfonates etc) under nucleophilic substitution reaction conditions to give phosphonate esters. For example prodrugs of formula 1 where R1 is acyloxymethyl group can be synthesized through direct alkylation of the free phosphonic acid of formula 5, with the desired acyloxymethyl halide (e.g. Cl, Br, I; Elhaddadi, et al Phosphorus Sulfur, 1990, 54(1-4): 143; Hoffmann, Synthesis, 1988, 62) in presence of base e.g. N,N_-dicyclohexyl-4-morpholinecarboxcamidine, Hunigs base etc. in polar aprotic solvents such as DMF (Starrett, et al, J. Med. Chem., 1994, 1857). These carboxylates include but not limited to acetate, propylate, isobutyrate, pivalate, benzoate, and other carboxylates. Alternately, these acyloxymethylphosphonate esters can also be synthesized by treatment of the nitrophosphonic acid (A is NO2 in formula 5; Dickson, et al, J. Med. Chem., 1996, 39: 661; Iyer, et al, Tetrahedron Lett., 1989, 30: 7141; Srivastva, et al, Bioorg. Chem., 1984, 12: 118). This can be extended to many other types of prodrugs, such as compounds of formula 1 where R1 is 3-phthalidyl, 2-oxo-4,5-didehydro-1,3-dioxolanemethyl, and 2-oxotetrahydrofuran-5-yl groups, etc. (Biller and Magnin (U.S. Pat. No. 5,157,027); Serafinowska et al. (J. Med. Chem. 38: 1372 (1995)); Starrett et al. (J. Med. Chem. 37: 1857 (1994)); Martin et al. J. Pharm. Sci. 76: 180 (1987); Alexander et al., Collect. Czech. Chem. Commun, 59: 1853 (1994)); and EPO 0632048A1). N,N-Dimethylformamide dialkyl acetals can also be used to alkylate phosphonic acids (Alexander, P., et al Collect. Czech. Chem. Commun., 1994, 59, 1853).

Alternatively, these phosphonate prodrugs or phosphoramidates can also be synthesized, by reaction of the corresponding dichlorophosphonates and an alcohol or an amine (Alexander, et al, Collect. Czech. Chem. Commun., 1994, 59: 1853). For example, the reaction of dichlorophosphonate with phenols and benzyl alcohols in the presence of base (such as pyridine, triethylamine, etc) yields compounds of formula 1 where R1 is aryl (Khamnei, S., et al J. Med. Chem., 1996,39: 4109; Serafinowska, H. T., et al J. Med. Chem., 1995, 38: 1372; De Lombaert, S., et al J. Med. Chem., 1994,37: 498) or benzyl (Mitchell, A. G., et al J. Chem. Soc. Perkin Trans. 1, 1992, 38: 2345). The disulfide-containing prodrugs, reported by Puech et al., Antiviral Res., 1993, 22: 155, can also be prepared from dichlorophosphonate and 2-hydroxyethyl disulfide under the standard conditions.

Such reactive dichlorophosphonate intermediates, can be prepared from the corresponding phosphonic acids and the chlorinating agents e.g. thionyl chloride (Starrett, et al, J. Med. Chem., 1994, 1857), oxalyl chloride (Stowell, et al, Tetrahedron Lett., 1990, 31: 3261), and phosphorus pentachloride (Quast, et al, Synthesis, 1974, 490). Alternatively, these dichlorophosphonates can also be generated from disilyl phosphonate esters (Bhongle, et al, Synth. Commun., 1987, 17: 1071) and dialkyl phosphonate esters (Still, et al, Tetrahedron Lett., 1983, 24: 4405; Patois, et al, Bull. Soc. Chim. Fr., 1993, 130: 485).

Furthermore, these prodrugs can be prepared from Mitsunobu reactions (Mitsunobu, Synthesis, 1981, 1, Campbell, J. Org. Chem., 1992, 52: 6331), and other acid coupling reagents include, but not limited to, carbodiimides (Alexander, et al, Collect. Czech. Chem. Commun., 1994; 59: 1853; Casara, et al, Bioorg. Med. Chem. Lett., 1992, 2: 145; Ohashi, et al, Tetrahedron Lett., 1988, 29: 1189), and benzotriazolyloxytris-(dimethylamino)phosphonium salts (Campagne, et al, Tetrahedron Lett., 1993, 34: 6743). The prodrugs of formula 1 where R1 is the cyclic carbonate or lactone or phthalidyl can also be synthesized by direct alkylation of free phosphonic acid with desired halides in the presence of base such as NaH or diisopropylethylamine (Biller and Magnin U.S. Pat. No. 5,157,027; Serafinowska et al. J. Med. Chem. 38: 1372 (1995); Starrett et al. J. Med. Chem. 37: 1857 (1994); Martin et al. J. Pharm. Sci. 76: 180 (1987); Alexander et al., Collect. Czech. Chem. Commun, 59: 1853 (1994); and EPO 0632048A1).

R1 can also be introduced at an early stage of synthesis, when feasible. For example, compounds of formula I where R1 is phenyl can be prepared by phosphorylation of 2,5-anhydro-6-O-(t-butyldiphenylsilyl)-1-1deoxy-1,1-difluoro-3,4-O-isopropylidene-D-ribo-hex-1eniol via strong base treatment (e.g. BU₃SnH) followed by phenylseleniumdiethylphosphonate as shown in the following scheme.

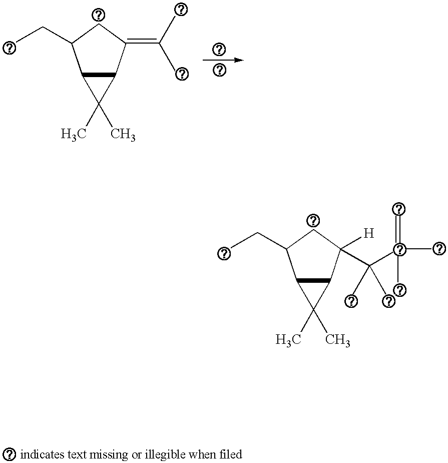

It is envisioned that compounds of formula I can be mixed phosphonate esters by combining the above described prodrugs (e.g. phenyl benzyl phosphonate esters, phenyl acyloxyalkyl phosphonate esters, etc.). For example, the chemically combined phenyl-benzyl prodrugs are reported by Meier et al. Bioorg. Med. Chem. Lett., 1997; 7: 99.

The substituted cyclic propyl phosphonate esters of formula 5, can be synthesized by reaction of the corresponding dichlorophosphonate and the substituted 1,3-propanediol. The following are some of the methods to prepare the substituted 1,3-propanediols.

Synthesis of the 1,3-Propanediols Used in the Preparation of Certain Prodrugs

The discussion of this step includes various synthetic methods for the preparation of the following types of propane-1,3-diols: i) 1-substituted; ii) 2-substituted; and iii) 1,2- or 1,3-annulated. Different groups on the prodrug part of the molecule i.e., on the propanediol moiety can be introduced or modified either during the synthesis of the diols or after the synthesis of the prodrugs.

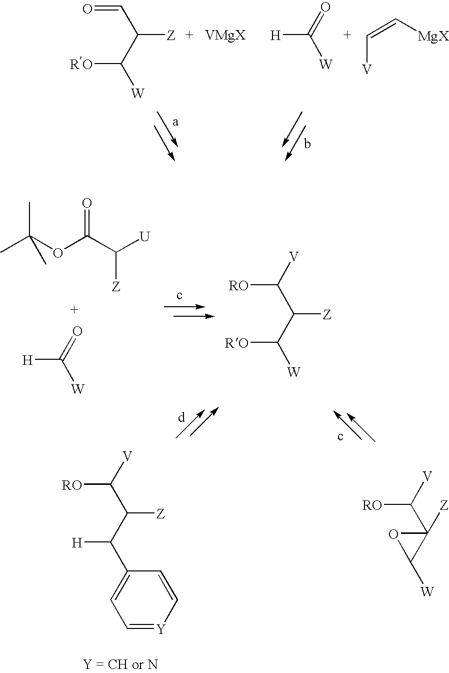

### i) 1-Substituted 1.3-Propanediols

Propane-1,3-diols can be synthesized by several well known methods in the literature. Aryl Grignard additions to 1-hydroxypropan-3-al gives 1-aryl-substituted propane-1,3-diols (path a). This method will enable conversion of various substituted aryl halides to, 1-arylsubstituted-1,3-propanediols (Coppi, et. al., J. Org. Chem., 1988, 53, 911). Aryl halides can also be used to synthesize 1-substituted propanediols by Heck coupling of 1,3-diox-4-ene followed by reduction and hydrolysis (Sakamoto, et. al., Tetrahedron Lett., 1992,33, 6845). A variety of aromatic aldehydes can be converted to 1-substituted-1,3-propanediols by vinyl Grignard addition followed by hydroboration (path b). Substituted aromatic aldehydes are also utilized by lithiuim-t-butylacetate addition followed by, ester reduction (path e) (Turner., J. Org. Chem., 1990, 55 4744). In another method, commercially available cinnamyl alcohols can be converted to epoxy alcohols under catalytic asymmetric epoxidation conditions. These epoxy alcohols are reduced by Red-AI to result in enantiomerically pure propane-1,3-diols (path c). Alternatively, enantiomerically pure 1,3-diols can be obtained by chiral borane reduction of hydroxyethyl aryl ketone derivatives (Ramachandran, et. al., Tetrahedron Lett., 1997, 38 761). Pyridyl, quinoline, and isoquinoline propan-3-ol derivatives can be oxygenated to 1-substituted propan-1,3-diols by N-oxide formation followed by rearrangement under acetic anhydride conditions (path d) (Yamamoto, et. al., Tetrahedron, 1981, 37, 1871).

### ii) 2-Substituted 1,3-Propanediols

Various 2-substituted propane-1,3-diols can be made from commercially available 2-(hydroxymethyl)-1,3-propanediol. Triethyl methanetric arboxylate. can be converted to the triol by complete reduction (path a) or diol-monocarboxylic acid derivatives can be obtained by partial hydrolysis and diester reduction (Larock, Comprehensive Organic Transformations, VCH, New York, 1989). Nitrotriol is also known to give the triol by reductive elimination (path b) (Latour, et. al., Synthesis, 1987, 8, 742). The triol can be derivatized as a mono acetate or carbonate by treatment with alkanoyl chloride, or alkylchoroformate, respectively (path d) (Greene and Wuts, Protective Groups in Organic Synthesis, John Wiley, New York, 1990). Aryl substitution can be made by oxidation to the aldehyde followed by aryl Grignard additions (path c) and the aldehyde can also be converted to substituted amines by reductive amination reactions (path e). ##STR20##

### iii) Annulated 1,3-Propanediols

Prodrugs of formula 1 where V-Z or V—W are fused by three carbons are made from cyclohexanediol derivatives. Commercially available cis, cis-1,3,5-cyclohexanetriol can be used for prodrug formation. This cyclohexanetriol can also be modified as described in the Ocase of 2-substituted propane-1,3-diols to give various analogues. These modifications can either be made before or after formation of prodrugs. Various 1,3-cyclohexanediols can be made by Diels-Alder methodology using pyrone as the diene (Posner, et al., Tetrahedron Lett., 1991, 32, 5295). Cyclohexyl diol derivatives are also made by nitrile oxide olefin-additions (Curran, et. al., J. Am. Chem. Soc., 1985, 107, 6023). Alternatively, cyclohexyl precursors can be made from quinic acid (Rao, et. al., Tetrahedron Lett., 1991, 32, 547.)

(2) Deprotection of Phosphonate Esters

Compounds of formula 1 where R1 is H may be prepared from phosphonate esters using known phosphate and phosphonate ester cleavage conditions. For example, alkyl phosphonate esters are generally cleaved by reaction with silyl halides followed by hydrolysis of the intermediate silyl phosphonate esters. Depending on the stability of the products, these reactions are usually accomplished in the presence of acid scavengers such as 1,1,1,3,3,3-hexamethyldisilazane, 2,6-lutidine, etc. Various silyl halides can be used for this transformation, such as chlorotrimethylsilane (Rabinowitz J. Org. Chem., 1963, 28: 2975), bromotrimethylsilane (McKenna et al. Tetrahedron Lett., 1977, 155), iodotrimethylsilane (Blackburn et al. J. Chem. Soc., Chem. Commun., 1978, 870). Phosphonate esters can also be cleaved under strong acid conditions, such as hydrogen halides in acetic acid or water, and metal halides (Moffatt et al. U.S. Pat. No. 3,524,846, 1970). Phosphonate esters can also be converted to dichlorophosphonates with halogenating agents (e.g. PCl₅, SOCl₂, BBr₃, etc. Pelchowicz et al. J. Chem. Soc., 1961, 238) and subsequently hydrolyzed to give phosphonic acids. Reductive reactions are useful in cleaving aryl and benzyl phosphonate esters. For example, aryl and benzyl phosphonate esters can be cleaved under hydrogenolysis conditions (Lejczak et al. Synthesis, 1982, 412; Elliott et al. J. Med Chem., 1985, 28: 1208.) or dissolving metal reduction conditions (Shafer et al. J. Am. Chem. Soc., 1977, 99: 51 18). (Elliott et al. J. Med. Chem., 1985,28: 1208). Electrochemical (Shono et al. J. Org. Chem., 1979, 44: 4508) and pyrolysis (Gupta et al. Synth. Commun., 1980, 10: 299) conditions have also been used to cleave various phosphonate esters.

The synthesis of compounds such as those found in formula I may, depending on the route of synthesis, produce anomeric mixtures. These mixtures may be separated and the anomers are denoted as Ia in which X is below the plane of the ribose ring or Ib in which X is above the plane of the ribose ring.

The synthesis of ribosyl phosphoramidates utilizes the Staudinger reaction (Casero F, Cipolla L, Lay L, Nicotra F, Panza L and Russo G, J. Org. Chem. 61, 3428 (1981)) of suitably protected ribosyl azides with trimethyl phosphite followed by deprotection.

As a way of treating either type II diabetes or cancer or any metabolic inbalance, the above mentioned compounds may be formulated in a drug delivery system to a human in order to cure or control either disease. These formulations may include but are not limited to pills, patches, injections or inhalants. Compounds of the invention are administered orally in a total daily dose of about 0.1 mg/kg/dose to about 100 mg/kg/dose, preferably from about 0.3 mg/kg/dose to about 30 mg/kg/dose. The most preferred dose range is from 0.5 to 10 mg/kg (approximately 1 to 20 nmoles/kg/dose). The use of time-release preparations to control the rate of release of the active ingredient may be preferred. The dose may be administered in as many divided doses as is convenient. When other methods are used (e.g. intravenous administration), compounds are administered to the affected tissue at a rate from 0.3 to 300 nmol/kg/min, preferably from 3 to 100 nmoles/kg/min. Such rates are easily maintained when these compounds are intravenously administered as discussed below.

For the purposes of this invention, the compounds may be administered by a variety of means including orally, parenterally, by inhalation spray, topically, or rectally in formulations containing pharmaceutically acceptable carriers, adjuvants and vehicles. The term parenteral as used here includes subcutaneous, intravenous, intramuscular, and intraarterial injections with a variety of infusion techniques. Intraarterial and intravenous injection as used herein includes administration through catheters. Oral administration is generally preferred.

Pharmaceutical compositions containing the active ingredient may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient, which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents; such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium such as peanut oil, liquid paraffin or olive oil.

Aqueous suspensions of the invention contain the active materials in admixture with excipients suitable for the manufacture of aqueous-suspensions. Such excipients include a suspending agent, such as sodium carboxymethylcellulose, methylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

Oil suspensions may be formulated by suspending the active ingredient in a vegetable oil, such as arachis oil, olive oil, sesame oil or coconut oil, or ia mineral oil such as liquid paraffin. The oral suspensions may contain a thickening agent, such as beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

Dispersible powders and granules of the invention suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the invention may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion may also contain sweetening and flavoring agents.

Syrups and elixirs may be formulated with sweetening agents, such as glycerol, sorbitol or sucrose. Such formulations may also contain a demulcent, a preservative, a flavoring or a coloring agent.

The pharmaceutical compositions of the invention may be in the form of a sterile injectable preparation, such as a sterile injectable aqueous or oleaginous suspension. This suspension may be formulated according to the known art using those suitable dispersing or wetting agents and suspending agents, which have been mentioned above. The sterile injectable preparation may also be a sterile injectable solution or suspension in a non-toxic parenterally acceptable diluent or solvent such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium For this purpose any bland fixed oil may be employed including synthetic mono- or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, a time-release formulation intended for oral administration to humans may contain 20 to 2000 mmol (approximately 10 to 1000 mg) of active material compounded with an appropriate and convenient amount of carrier material-which may vary from about 5 to about 95% of the total compositions. It is preferred that the pharmaceutical composition be prepared which provides easily measurable amounts for administration. For example, an aqueous solution intended for intravenous infusion should contain from about 0.05 to about 50 mmol (approximately 0.025 to 25 mg) of the active ingredient per milliliter of solution in order that infusion of a suitable volume at a rate of about 30 mL/hr can occur.

As noted above, formulations of the present invention suitable for oral administration may be presented as discrete units such as capsules, cachets or tablets each containing a predetermined amount of the active ingredient, as a powder or granules; as a solution or a suspension in an aqueous or non-aqueous liquid, or as an oil-in-water liquid emulsion or a water-in-oil liquid emulsion. The active ingredient may also be administered as a bolus, electuary or paste.

A tablet may be made by compression or molding, optionally with one or more accessory ingredients. Compressed tablets may be prepared by compressing in a suitable machine the active ingredient in a free flowing form such as a powder or granules, optionally mixed with a binder (e.g., povidone, gelatin, hydroxypropyl ethyl cellulose), lubricant, inert diluent, preservative, disintegrant (e.g., sodium starch glycolate, cross-linked povidone, cross-linked sodium carboxymethyl cellulose) surface active or dispersing agent. Molded tablets may be made by molding, in a suitable machine, a mixture of the powdered compound moistened with an inert liquid diluent. The tablets may optionally be coated or scored and may be formulated so as to provide. slow or controlled release of the active ingredient therein using, for example, hydroxypropyl methylcellulose in varying proportions to provide the desired release profile. Tablets may optionally be provided with an enteric coating, to provide release in parts of the gut other than the stomach. This is particularly advantageous with the compounds of formula I when such compounds are susceptible to acid hydrolysis.

Formulations suitable for topical administration in the mouth include lozenges comprising the active ingredient in a flavored base, usually sucrose and acacia or tragacanth; pastilles comprising the active ingredient in an inert base such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous isotonic sterile injection solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Injection solutions and suspensions may be prepared from sterile powders, granules and tablets of the kind previously described.

Preferred unit dosage formulations are those containing a daily dose or unit, daily sub-dose, or an appropriate fraction thereof, of a modulator of glucose metabolism.

It will be understood, however, that the specific dose level for any particular patient will depend on a variety of factors including the activity of the specific compound employed, the age, body weight, general health, sex and diet of the individual being treated; the time and route of administration; the rate of excretion; other drugs which have previously been administered; and the severity of the particular disease undergoing therapy, as is well understood by those skilled in the art.

Utility:

The Utility of Compound 2 as a Treatment for Diabetes is Shown in the Following Data:

Studies of Compound 2 have shown that it activates 6-phosphofructo-1-kinase (6PF1K) and inhibits fructose-1,6-bisphosphatase (FBPase). Additionally, in vitro studies have shown the compound 2, when incubated with FAO hepatoma cells, inhibits glucose production. Given the dual functionality of compound 2, it is possible to account for the inhibition of glucose production in FAO cells by assuming that it directly inhibits FBPase or that by activating 6PF1K, compound 2 is increasing the rate of glycolysis. It is well known that by increasing the rate of glycolysis, the rate of gluconeogenesis decreases (Hanson et al. (1984) J. Biol. Chem. 259, 218-223).

Ka is the concentration of substrate giving half-maximal activation.

TABLE 2
Activation of 6-phosphofructo-1-kinase by Compound 2.1

| Substrate | Ka (μM) |
|---|---|
| fructose-2,6-bisphosphate | 0.09 |
| ribose-1,5-bisphosphate | 3.5 |
| Compound 2 | 6.5 |

TABLE 3
Inhibition of Fructose-1,6-bisphosphatase by Compound 2.1

| | | Maximum |
|---|---|---|
| | | Decrease in |
| | K_{I} (μM) | Activity (%) |
| At 5 μM Fru-1,6- | | |
| bisphosphate | | |
| Rib-1,5-P₂ | 87 | 50 |
| Compound 2.1 | 87 | 50 |
| At 20 μM Fru-1,6- | | |
| bisphosphate | | |
| Rib-1,5-P₂ | 87 | 50 |
| Compound 2.1 | 87 | 50 |

TABLE 4
Inhibition of Glucose Production in FAO cells by Compound 2.1

| Ia (μM) | Percent inhibition of glucose production |
|---|---|
| 0 | 0 |
| 60 | 20 ± 15 |
| 125 | 35 ± 10 |
| 250 | 40 ± 10 |
| 500 | 35 ± 10 |
| 100 | 60 ± 15 |

The Utility of Compound 1.1 as a Treatment for Diabetes is Shown in the Following Data:

Glucose Tolerance Test

Compound 1.1 was shown to reduce blood glucose levels in diabetic Zucker (fa/fa) that were fasted, injected with compound 1.1 and then challenged with an infusion of glucose.

Thirty-six 16 wk old male Zucker (fa/fa) rats were randomly assigned to one of the following groups:
1. Vehicle (0.9% NaCl)
2. Metformin—320 mg/kg
3. Compound 1.1—10 mg/kg Acid
4. Compound 1.1—100 mg/kg Acid
5. Compound 3.1—10 mg/kg Ester
6. Compound 3.1—100 mg/kg Ester

Animals were fasted for 16-18 hr and lightly anesthetized with a mixture of ketamine/xylazine at a dose of 0.5 ml/kg (45 mg/kg ketamine, 5 mg/kg xylazine). Animals received their respective treatments via IP injection (1.5 ml/kg) followed by an IP injection of glucose at 1.5 g/kg. Blood glucose was measured at 0, 30, 60, 90, and 120 minutes post-glucose.

TABLE 2

| | Blood Glucose (mg/dL) |
|---|---|
| Agent | at 120 minutes |
| Vehicle (0.9% NaCl) | 500 |
| Metformin - 320 mg/kg | 400 |
| Compound 1.1 - 10 mg/kg Acid | 460 |
| Compound 1.1 - 100 mg/kg Acid | 400 |
| Compound 2.1 - 10 mg/kg Ester | 500 |
| Compound 2.1 - 100 mg/kg Ester | 500 |

The Utility of Compounds 3.1 and 4.1 as Stimulators of Insulin Secretion is Shown in the Following Data:

Stimulation of Insulin Secretion

INS-1 clonal pancreatic B-cells were grown in well plates or T-25 flasks incubated overnight with INS-1 cell media with 3 mM glucose and 500 μM of compound 3 or compound 4. Insulin samples were collected for 10 minutes at 18 second intervals. Insulin release was greater for both compound 3.1 and compound 4.1 compared to controls, by about 50 percent, under these conditions. At 16 mM glucose the activating effect disappeared.

The Utility of Compound 4.1 as an Inhibitor of Lipid Levels in Liver Cells is Shown in the Following Data:

Inhibition of Intracellular Lipid Synthesis

The utility of compound 4.1 as treatment for the reduction of intracellular lipids is shown by the following data: incubation of 500 μM compound 4 in HTC hepatoma cells overnight resulted in a 26% decrease in triglyceride levels and near 20% decrease in cholesterol levels compared to control. This effect was quantitatively similar to the effect that 500 μM metformin had on these cells.

| | Triglyceride | Cholesterol |
|---|---|---|
| | (μg/mg protein) | (μg/mg protein) |
| Control | 60 | 48 |
| 500 μM Metformin | 42 | 38 |
| 500 μM Compound 4.1 | 44 | 39 |

The Utility of Compound 4.1 as an Inhibitor of cPNP is Shown in the Following Data:

Inhibition of Calf Spleen Purine Nucleoside Phosphorylase (cPNP)

Compound 4.1 (27 μM) was incubated with calf spleen purine nucleoside phosphorylase with increasing concentrations of inorganic phosphate. The results are shown below indicate that compound is likely a competitive inhibitor of cPNP:

| Pi (μM) | Control | Compound 4.1 |
|---|---|---|
| 20 | 170* | 18* |
| 50 | 237 | 31 |
| 200 | 298 | 96 |
| 1500 | 327 | 176 |

*activity in arbitrary units

The Utility of Compound 4.1 as an Anticancer Agent is Shown in the Following Data:

| Compound | cell line | IC50 |
|---|---|---|
| 1.1 | HL-60, Leukemia | 170 μM |
| 3.1 | NCI-H226 | 10(−7)M |
| | NCI/ADR-RES | 10(−7)M |
| | OVCAR-8 | 10(−8)M |
| | HT-29 | 10(−7)M |
| 4.1 | Rh1, Ewing Sarcoma | 170 μM |

### EXAMPLE 1

### Synthesis of Ribose-1-methylenediethylphosphonate

A solution of 15 g of dried D-ribose and 210 mg of p-toluene sulfonic acid(PTSA) and 40 ml of 2,2-dimethoxypropane in 160 ml N,N dimethylformamide(DMF) is stirred for 3 h at room temperature. An excess of AMBERLITE IRA-410(OH− form) was added to neutralize the acid. The resin was dried to prevent possible hydrolysis of product. The resin was filtered after two hours. The DMF was removed in vacuo at 60° C. and the syrup was chromatographed on silica gel (250 g) using CHCl₃/CH₂Cl₂(3:1). The Hasegawa paper used silicic acid CHCl₃/ethanol (50:1). The product is 2,3-O isopropylidene-D-ribofuranose (2a,2b).

The product was dissolved in pyridine and an equal-molar amount of pivaloyl chloride was added. The reaction was stirred overnight. The solvent was removed and the product 5-pivaloyl 2,3-O isopropylidene-D-ribofuranose (3a, 3b) was purified on silica gel.

Tetraethylenemethylenebisphosphonate (TEMBP) was dissolved in dry THF and an equal-molar amount of NaH was added to the solution. After 30 minutes an equal-molar quantity of 3a.3b dissolved in THF was added all at once and the mixture was stirred for 1.5 hr.

The reaction was stopped with NH₄OAC(aq) The THF was removed under vacuum and chloroform was added. The mixture was poured into a separatory flask and the organic layer was recovered. The organic layer was dried over MgSO₄. The chloroform was removed and the residue was placed on a silica gel column and the product was purified.(4a,4b)

To dry ethyl alcohol, NaOEt was added. 4a, 4b was dissolved in ethyl alcohol and added to the former solution. After 1.5 hr the reaction was stopped with water. The alcohol was removed and the residue was dissolved in chloroform. This solution was placed in a separatory flask and washed with NH₄OAC(aq). The organic layer was recovered, dried and the solvent removed. The residue was purified on silica gel resulting in the separation of the two anomers 5a and 5b.

Sa was dissolved in methanol. A 20 fold(w/w) excess of Dowex AG 50 (H+ form) resin was added and the reaction was left overnight. 1H-NMR confirmed the removal of the acetonide group giving 6a. 1H-NMR (CDCl₃) δ: 4.2 (m, 1H), 4.0 (m, 3H), 3.75(dd, 1H), 3.7(m, 4H), 3.6(dd, 1H), 2.2(m, 2H), 1 3(t, 6H); ¹³C-NMR (CDCl₃) δ: 28.81, 72.53, 77.63, 77.63, 80.44, 64.97, 51.10 and 52.60 (OCH₂CH₃), 61.56 and 62.34 (OCH₂CH₃); ³¹P-NMR (D₂O) δ: 20.136 (s, 1P)

Compound 5b was dissolved in methanol. A 20 fold(w/w) excess of Dowex AG 50 (H+ form) resin was added and the reaction was left overnight. 1H-NMR confirmed the removal of the acetonide group giving 6b. 1H-NMR (CDCl₃) δ: 4.1 (m, 1H), 3.9 (m, 1H), 3.8-3.4 (m, 8H), 2.2(m, 2H), 1.3 (t, 6H); ¹³C-NMR (CDCl₃) δ: 37.53, 79.36, 75.58, 783.15, 86.85, 68.51, 52.07 and 52.69 (OCH₂CH₃), 63.44 and 64.27 (OCH₂CH₃); ³¹P-NMR (D₂O) δ: 19.188 (s, 1P)

### EXAMPLE 2

### Synthesis of the difluoromethylene phosphonate

2,3-isopropylidine-α-D-ribonolactone (2): Starting with commercially available α-D-ribonolactone (10.0 g, 67 mmol) and p-toluenesulfonic acid (200 mg) was dissolved in anhydrous acetone (150 ml) and cooled to 0-C. 2,2-Dimethoxy propane (7.55 g, 80.4 mmol) was added to the reaction slowly over a period of 5 minutes. The reaction was stirred for 2 hours at room temperature then sodium bicarbonate powder (250 mg) was added to the reaction, stirred for five minutes, filtered and concentrated. Column chromatography of the crude material gave 2,3-isopropylidine-α-D-ribonolactone(1.06 g, 82%).

5-O-(t-butyldiphenylsilyl)-2,3-isopropylidine-α-D-ribonolactone (3): Compound 2 (19 g, 0.1 mol) and imidazole were dissolved in dichlorobenzene (100 ml) and cooled to −10 C. Then a solution of t-butyldiphenylsilylchloride (33.3 g, 0.12 mol) in dichloromethane (100 ml) was added to the rection mixture slowly over a period of 10 minutes. The reaction was stirred at room temperature for a period of 5 hours and then the solid was filtered off and the filtrate concentrated. Chromatography of the crude product yielded a colorless liquid (35 g, 83%).

2,5-anhydro-6-O-(t-butyldiphenylsilyl)-1deoxy-1,1-difluoro-3,4-O-isopropylidene-D-ribo-hex-1-eniol (4): To a solution of 3 (2.0 g,4.691 mmol) in tetrahydrofuran (75 ml), cooled to −20 C, was added dibromodifluoromethane (23 ml, 23.5 mmol) using a cooled syringe. To the vigorously stirred solution was added tris(dimethylamino)phosphene (8.5 ml, 46.9 mmol) after which a dense white precipitate formed immediately. The mixture was stirred at room temperature for 30 minutes and then a vacuum applied for another thirty minutes. Argon was then passed into the reaction vessel for five minutes. Tris(dimethylamino)phosphene (200 uL) was added followed by zinc powder (3.04 g, 46.9 mmol), both added to the reaction mixture which was heated to reflux for fifteen hours. The reaction mixture turned dark brown and was allowed to cool to room temperature. Diethyl ether (50 ml)was then added. The ether layer was decanted and the residue was washed with ether (30 ml) three times. The combined ether layers were washed with saturated copper sulfate solution followed by water and brine. The product was concentrated and chromatographed yielding compound 4 as a colorless oil. 1HNMR δ: 1.02(s, 9H), 1.40 (s,3H), 1.49 (s,3H), 3.75 (dd, J=19.5, 3.6, 1H), 3.85 (dd, J=19.5, 3.6, 1H), 4.45 (bs,1H), 4.91 (m, 1H), 5.40 (m, 1H), 7.39-7.48 (m,6H), 7.60-7.65(4H). 13C: d: 135.57, 135.54,130.07,130.01,127.95,127.93, 113.12,87.35, 81.58,78.41 ,65.04,26.85,26.65,25.75,19.07.

Diethyl (2,3-O-isopropylidene-5-Otert-butyldiphenylsilanyl)-D-ribofuranos-1yl)difluoromethylenephosphonate (5): A solution of 4 (2.0 g, 4.35) and diethyl(phenylselenyl)phosphonate (3.8 g,13.05 mmol) in dry benzene (10 ml) was degassed at reflux four one hour. To this refluxing solution was added a solution of AIBN (0.5 mmol) and tri-butyltin hydride (73 ml, 4.0 mmol) in dry degassed benzene (3.5 ml) over ten hours via syringe pump. The mixture was refluxed for four hours after the addition and concentrated. Chromatography of the crude gave a colorless oil (0.6 g, 27%). 1H-NMR δ: 1.05 (s, 9H), 1.25-1.39 (m, 9H), 1.54 (s, 3H), 3.68-3.78 (m, 2H), 4.18-4.32 (m, 5H), 4.35-4.43 (m, 1H), 4.60 (m, 1H), 4.94 (m, 1H), 7.4 (m, 6H), 7.68 )m, 4H); 13CNMR δ: 135.65,135.63,135.61,135.58,133.15,133.12,129.85,129.83,127.98,127. 97,127.80,127.78,11433,85.93,81.67,64.77,64.72,63.66,27.39,26.84,26.80,25.45,19.24,1 6.42,16.38,16.34; 19FNMR d: −118.25,−121.27; 31PNMR d: 5.5 (t, 1P).

Diethyl (D-ribo-furanos-1-yl)difluoromethylenephosphonate (6): A solution of 5 (2.7 g. 4/51 mmol) mixed in with a 2:1 ratio of trifluoroacetic acid and water (10 ml) was refluxed for six hours and then concentrated to dryness. The crude product was purified by silca gel col . . . Mn chromatography, yielding diethyl-D-ribo-furanos-1yl) difluoromethylene phosphonate (900 mg, 74%). [α]²⁵_{D}+3.69 (con. 0.179 M in MeOH). 1HNMR (CDCl₃) δ: 1.4 (m, 6H), 1.78 (bs, 1H, OH), 3.65 (dd, J=27.9, 3H, H+2OH), 3.65 (d, J=7, 1H), 3.88 (d, J=22), 4.04 (d, J=1 1), 4.60 (m, 6H), 4.90 (m, 1H); ¹³C-NMR (CDCl₃) d: 85.4,71.71,71.24,66.85,6632,61.56, 17.23; ¹⁹F-NMR −118 (d, 1F), −124 (d, IF); ³¹P δ: 5.5 (t, 1P) High Resolution Mass Spec: 321.0904 (expected: 321.0960); elemental analysis: C 37.55, H 6.17; expected C 37.51, H 5.98.

D-ribo-furanos-1-yl-difluoromehtylenephosphonate (7): A solution of 5 (200 mg, 0.33 mmol) in dimethyl formamide (5 ml) was cooled to 10° C. and trimethylsilylchloride was added in excess (1 ml). The reaction was heated for ten hours at 60° C. The reaction was concentrated and recrystallized from a ethyl acetate and methanol (3:1) mixture to yield D-ribo-furanos-1-yl-difluoromethylenephosphonate. (80 mg, 75%) [α]²⁵_{ZD} +6.92 (con. 0.133M in MeOH). 1HNMR (CDCl₃) δ: 3.61 (dd, J=11,11,1H), 3.82 (d,J=23, 1H), 3.6 (m, 1H), 4.05 (t, J=12), 4.2-4.7 (m, 1H), 4.05 (m, 1H); ¹³C-NMR (CD₃OD) δ: 83.41, 71.02, 70.71, 66.85,66.32,61.53; ¹⁹F-NMR −119.24 (d, 1F), −125.05 (d, 1F); 31P d: 5.5 (t, 1P); ³¹P δ: 2.4 (t, 1P); High Resolution Mass Spec: 265.9985 (required 265.9290); elemental analysis: C 26.98, H 4.15; required C 27.28, H 4.20.

Compounds prepared from literature sources (Meyer, R B, et al., (1984) J. Med. Chem. 27,1095-1098, and Linn, G. (1993) Synthesis of Phosphonate Analogues of Ribose-1-5-bisphosphate, Ph.D. thesis, City University of New York:

1.1 ribose-1-methylenephosphonate: Formula Ia, where X═CH₂, A=CH₂OH, B═OH, R1=H

2.1 5-phosphoribosyl-1-methylenephosphonate: Formula Ia, where X═CH₂, A=CH₂OPO(OH)₂, B═OH, R1=H

Compounds Made from This Invention:

3.1 ribose-1-methylenediethylphosphonate: Formula Ia, where X═CH₂, A=CH₂OH, B═OH, R1=Ethyl

4.1 ribose-1-difluoromethylenephosphonate: Formula Ia, where X═CF₂, A=CH₂OH, B═OH, R1=H

What is claimed is:

1. A compound having the formula as shown: 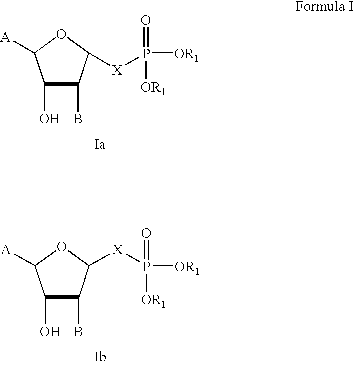
where X is selected from the group consisting of —CH₂, CHF, CF₂, S NH, alkyl, alkenyl, alkynyl, alkyl(carboxyl), alkyl(hydroxy), alkyl(phosphonate), alkyl(sulfonate, aryl, alkylaminoalkyl, alkoxyalkyl, alkylthioalkyl, alkylthio, alicyclic, 1-monohaloalkyl, 1,1 dihaloalkyl, carbonylalkyl, aminocarbonylamino, alkylaminocarbonyl, alkylcarbonylamino, aralkyl and alkylaryl, all optionally substituted; A is (CH₂)ₙ-A′ where n is from 1-4 and A′ is hydroxy, halogen, OPO(OH)₂, alkyl, alkoxy, amino, azido or alkenyl; R1 is independently selected from the group consisting of alkyl, aryl, heteroalicyclic where the cyclic moiety contains a carbonate or thiocarbonate, —C(R²)₂-aryl, -alk-aryl, —C(R²)₂OC(O)NR²₂, —NR²—C(O)—R³, —C(R²)₂—OC(O)R³, —C(R²)₂—O—C(O)OR³, —C(R²)₂OC(O)SR³, -alk-S—C(O)R³, -alk-S—S-alkylhydroxy, and -alk-S—S—S-alkylhydroxy, or together R1 and R1 are -alk-S—S-alk- to form a cyclic group, wherein each “alk” is an independently selected alkylene, or together R1 and R1 are 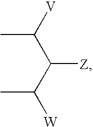
wherein
V and W are independently selected from the group consisting of hydrogen, aryl, substituted aryl, heteroaryl, substituted heteroaryl, 1-alkenyl, 1-alkynyl, and —R⁹; or
together V and Z are connected via a chain of 3-5 atoms, only one of which can be a heteroatom, to form part of a cyclic group substituted with hydroxy, acyloxy, alkoxycarboxy, or aryloxycarboxy attached to a carbon atom that is three atoms from an oxygen attached to the phosphorus; or
together V and W are connected via a chain of 3 carbon atoms to form part of a cyclic group substituted with hydroxy, acyloxy, alkoxycarboxy, alkylthiocarboxy, hydroxymethyl, or aryloxycarboxy attached to a carbon atom that is three atoms from an oxygen attached to the phosphorus;
Z is selected from the group consisting of —CH₂ OH, —CH₂ OCOR³, —CH₂OC(O)R³, —CH₂OC(O)SR³, —CH₂OCO₂R³, —SR³, —S(O)R³, —CH₂ N₃, —CH₂ NR²₂, —CH₂AR, —CH(Ar)OH, —CH(CH═CR²R²)OH, —CH(C═CR²)OH, and —R²;
with the provisos that:
a) V, Z, W are not all —H; and
b) when Z is —R², then at least one of V and W is not —H or —R⁹;
R² is selected from the group consisting of R³ and —H;
R³ is selected from the group consisting of alkyl aryl, alicyclic, heteroalicyclic, and aralkyl;
R⁴ is independently selected from the group consisting of —H, lower alkyl, lower alicyclic, lower heteroalicyclic, lower aralkyl, and lower aryl;
R⁵ is selected from the group consisting of lower alkyl, lower aryl, lower aralkyl, lower alicyclic, and lower heteroalicyclic;
R⁶ is independently selected from the group consisting of —H, and lower alkyl;
R⁷ is independently selected from the group consisting of —H, lower alkyl, lower alicyclic, lower heteroalicyclic, lower aralkyl, lower aryl, and —C(O)R¹⁰;
R⁸ is independently selected from the group consisting of —H, lower alkyl, lower aralkyl, lower aryl, lower alicyclic, —(O)R¹⁰, or together said R⁸ groups form a bidendate alkylene;
R⁹ is selected from the group consisting of alkyl, aralkyl, alicyclic, and heteroalicyclic;
R¹⁰ is selected from the group consisting of —H, lower alkyl, —NH₂, lower aryl, and lower perhaloalkyl;
R¹¹ is selected from the group consisting of alkyl, aryl, —OH, —NH₂ and —OR³; and pharmaceutically acceptable prodrugs and salts thereof.

2. A compound having formula I 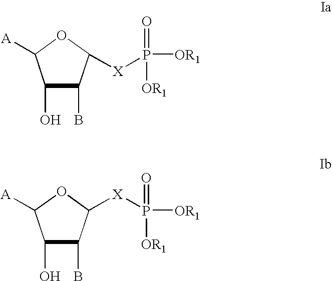
where X is selected from the group consisting of CHF, CF₂, S, NH, alkyl, alkenyl, alkynyl, alkyl(carboxyl), alkyl(hydroxy), alkyl(phosphonate), alkyl(sulfonate, aryl, alkylaminoalkyl, alkoxyalkyl, alkylthioalkyl, alkylthio, alicyclic, 1-monohaloalkyl, 1,1 dihaloalkyl, carbonylalkyl, aminocarbonylamino, alkylaminocarbonyl, alkylcarbonylamino, aralkyl and alkylaryl, all optionally substituted; A is (CH₂)ₙ-A′ where n is from 1-4 and A′ is hydroxy, halogen, OPO(OH)₂, alkyl, alkoxy, amino, azido or alkenyl; B is OH or F; R1 is independently selected from the group consisting of H, alkyl, aryl, heteroalicyclic where the cyclic moiety contains a carbonate or thiocarbonate, —C(R²)₂-aryl, -alk-aryl, —C(R²)₂ OC(O)NR²₂, —NR²—C(O)—R³, —C(R²)₂—OC(O)R³, —C(R²)₂—O—C(O)OR³, —C(R²)₂ OC(O)SR³, -alk-S—C(O)R³, -alk-S—S-alkylhydroxy, and -alk-S—S—S-alkylhydroxy, or together R1 and R1 are -alk-S—S-alk- to form a cyclic group, wherein each “alk” is an independently selected alkylene, or together R1 and R1 are 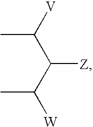
wherein
V and W are independently selected from the group consisting of hydrogen, aryl, substituted aryl, heteroaryl, substituted heteroaryl, 1-alkenyl, 1-alkynyl, and —R⁹; or
together V and Z are connected via a chain of 3-5 atoms, only one of which can be a heteroatom, to form part of a cyclic group substituted with hydroxy, acyloxy, alkoxycarboxy, or aryloxycarboxy attached to a carbon atom that is three atoms from an oxygen attached to the phosphorus; or
together V and W are connected via a chain of 3 carbon atoms to form part of a cyclic group substituted with hydroxy, acyloxy, alkoxycarboxy, alkylthiocarboxy, hydroxymethyl, or aryloxycarboxy attached to a carbon atom that is three atoms from an oxygen attached to the phosphorus;
Z is selected from the group consisting of —CH₂OH, —CH₂OCOR³, —CH₂OC(O)R³, —CH₂OC(O)SR³, —CH₂OCO₂R³, —SR³, —S(O)R³, —CH₂N₃, —CH₂NR²₂, —CH₂Ar, —CH(Ar)OH, —CH(CH═CR²R²)OH, —CH(C═CR²)OH, and —R²;
with the provisos that:
a) V, Z, W are not all —H; and
b) when Z is —R², then at least one of V and W is not —H or —R⁹;
R² is selected from the group consisting of R³ and —H;
R³ is selected from the group consisting of alkyl aryl, alicyclic, heteroalicyclic, and aralkyl;
R⁴ is independently selected from the group consisting of —H, lower alkyl, lower alicyclic, lower heteroalicyclic, lower aralkyl, and lower aryl;
R⁵ is selected from the group consisting of lower alkyl, lower aryl, lower aralkyl, lower alicyclic, and lower heteroalicyclic;
R⁶ is independently selected from the group consisting of —H, and lower alkyl;
R⁷ is independently selected from the group consisting of —H, lower alkyl, lower alicyclic, lower heteroalicyclic, lower aralkyl, lower aryl, and —C(O)R¹⁰;
R⁸ is independently selected from the group consisting of —H, lower alkyl, lower aralkyl, lower aryl, lower alicyclic, —(O)R¹⁰, or together said R⁸ groups form a bidendate alkylene;
R⁹ is selected from the group consisting of alkyl, aralkyl, alicyclic, and heteroalicyclic;
R¹⁰ is selected from the group consisting of —H, lower alkyl, —NH₂, lower aryl, and lower perhaloalkyl;
R¹¹ is selected from the group consisting of alkyl, aryl, —OH, —NH₂ and —OR³; and pharmaceutically acceptable prodrugs and salts thereof.

3. A method of treating hyperglycemia in an animal, which comprises administering to the animal a pharmaceutical composition of a compound having the formula 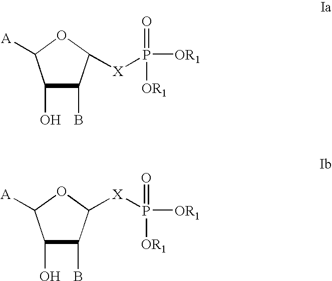
where X is selected from the group consisting of CH₂ CHF, CF₂, S, NH, alkyl, alkenyl, alkynyl, alkyl(carboxyl), alkyl(hydroxy), alkyl(phosphonate), alkyl(sulfonate, aryl, alkylaminoalkyl, alkoxyalkyl, alkylthioalkyl, alkylthio, alicyclic, 1-monohaloalkyl, 1,1 dihaloalkyl, carbonylalkyl, aminocarbonylamino, alkylaminocarbonyl, alkylcarbonylamino, aralkyl and alkylaryl, all optionally substituted; A is (CH₂)ₙ-A′ where n is from 1-4 and A′ is hydroxy, halogen, OPO(OH)₂, alkyl, alkoxy, amino, azido or alkenyl; B is OH or F; R1 is independently selected from the group consisting of H, alkyl, aryl, heteroalicyclic where the cyclic moiety contains a carbonate or thiocarbonate, —C(R²)₂-aryl, -alk-aryl, —C(R²)₂ OC(O)NR²₂, —NR²—C(O)—R³, —C(R²)₂—OC(O)R³, —C(R²)₂—0—C(O)OR³, —C(R²)₂ OC(O)SR³, -alk-S—C(O)R³, -alk-S—S-alkylhydroxy, and -alk-S—S—S-alkylhydroxy, or together R1 and R1 are -alk-S—S-alk- to form a cyclic group, wherein each “alk” is an independently selected alkylene, or together R1 and R1 are 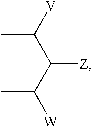
wherein
V and W are independently selected from the group consisting of hydrogen, aryl, substituted aryl, heteroaryl, substituted heteroaryl, 1-alkenyl, 1-alkynyl, and —R⁹; or
together V and Z are connected via a chain of 3-5 atoms, only one of which can be a heteroatom, to form part of a cyclic group substituted with hydroxy, acyloxy, alkoxycarboxy, or aryloxycarboxy attached to a carbon atom that is three atoms from an oxygen attached to the phosphorus; or
together V and Z are connected via a chain of 3-5 atoms, only one of which can be a heteroatom, to form part of a cyclic group substituted with hydroxy, acyloxy, alkoxycarboxy, or aryloxycarboxy attached to a carbon atom that is three atoms from an oxygen attached to the phosphorus; or
together V and W are connected via a chain of 3 carbon atoms to form part of a cyclic group substituted with hydroxy, acyloxy, alkoxycarboxy, alkylthiocarboxy, hydroxymethyl, or aryloxycarboxy attached to a carbon atom that is three atoms from an oxygen attached to the phosphorus;
Z is selected from the group consisting of —CH₂ OH, —CH₂ OCOR³, —CH₂OC(O)R³, —CH₂OC(O)SR³, —CH₂OCO₂R³, —SR³, —S(O)R³, —CH₂ N₃, —CH₂ NR²₂, —CH₂Ar, —CH(Ar)OH, —CH(CH═CR²R²)OH, —CH(C═CR²)OH, and —R²;
with the provisos that:
a) V, Z, W are not all —H; and
b) when Z is —R², then at least one of V and W is not —H or —R⁹;
R² is selected from the group consisting of R³ and —H;
R³ is selected from the group consisting of alkyl aryl, alicyclic, heteroalicyclic, and aralkyl;
R⁴ is independently selected from the group consisting of —H, lower alkyl, lower alicyclic, lower heteroalicyclic, lower aralkyl, and lower aryl;
R⁵ is selected from the group consisting of lower alkyl, lower aryl, lower aralkyl, lower alicyclic, and lower heteroalicyclic;
R⁶ is independently selected from the group consisting of —H, and lower alkyl;
R⁷ is independently selected from the group consisting of —H, lower alkyl, lower alicyclic, lower heteroalicyclic, lower aralkyl, lower aryl, and —C(O)R¹⁰;
R⁸ is independently selected from the group consisting of —H, lower alkyl, lower aralkyl, lower aryl, lower alicyclic, —(O)R¹⁰, or together said R⁸ groups form a bidendate alkylene;
R⁹ is selected from the group consisting of alkyl, aralkyl, alicyclic, and heteroalicyclic;
R¹⁰ is selected from the group consisting of —H, lower alkyl, —NH₂, lower aryl, and lower perhaloalkyl;
R¹¹ is selected from the group consisting of alkyl, aryl, —OH, —NH₂ and —OR³; and pharmaceutically acceptable prodrugs and salts thereof.

4. A method for treating hyperglycemia in an animal, which comprises administering to the animal, the pharmaceutical composition of claim 1.

5. A method for treating hyperglycemia in an animal, which comprises administering to the animal, the pharmaceutical composition of claim 2.

6. A pharmaceutical composition, which comprises the compounds of claim 1 in association with pharmaceutically acceptable diluent or carrier.

7. A pharmaceutical composition, which comprises the compounds of claim 3 in association with pharmaceutically acceptable diluent or carrier.

8. A pharmaceutical composition, which comprises the compound of claim 2 in association with a pharmaceutically acceptable diluent or carrier.

9. A method for treating cancer in an animal, which comprises administering to the animal a pharmaceutical composition of claim 1.

10. A method for treating cancer in an animal, which comprises administering to the animal, the pharmaceutical composition of claim 2.

11. A method for killing cancer cells, which comprises administering to the cells the compound of claim 1.

12. A method for killing cancer cells, which comprises administering to the cells, the compound of claim 2.

13. A method for killing cancer cell, which comprises administering to the cells the compound of claim 3.

14. A compound as in claim 1 which can be phosphorylated at the C(5) position.

15. A compound as in claim 2 which can be phosphorylated at the C(5) position.
